(19) United States
(12) Patent Application Publication
(10) Pub. No.: US 2005/0281774 A1
(43) Pub. Date: Dec. 22, 2005

(54) Use of polymers based on n-vinyl caprolactam

(75) Inventors: Gabi Muller, Mannheim (DE); Peter Hossel, Schifferstadt (DE); Tanja Schneider, Bensheim (DE); Claudia Wood, Weinheim (DE); Klemens Mathauer, Heidelberg (DE); Christian Drohmann, Brasschaat (BE); Son Nguyen-Kim, Hemsbach (DE)

Correspondence Address: CONNOLLY BOVE LODGE & HUTZ, LLP, P O BOX 2207, WILMINGTON, DE, 19899, US

(73) Assignee: BASF AKTIENGESELLSCHAFT, Ludwigshafen (DE)

(21) Appl. No.: 10/529,502

(22) PCT Filed: Sep. 18, 2003
(86) PCT No.: PCT/EP03/10373

§ 371 Date: Mar. 28, 2005

(30) Foreign Application Priority Data
Sep. 27, 2002 (DE) 102 45 586.4

(51) Int. Cl. A61K 7/06; A61K 7/11

(52) U.S. Cl. 424/70.15

(57) ABSTRACT

The present invention relates to cosmetic preparations, in particular for hair cosmetic applications, which comprise copolymers based on N-vinyllactams and N-vinyl heterocyclic compounds.

The present invention relates to cosmetic preparations, in particular for hair cosmetic applications, which comprise copolymers based on N-vinyllactams and N-vinyl heterocyclic compounds.

### PRIOR ART

Copolymers containing N-vinyllactam, such as Luviskol K, Luviskol VA, Luviquat Hold or Luviskol Plus (BASF), are used in cosmetic, in particular hair cosmetic, preparations, in particular as hair-setting compositions.

Gel preparations are used for hair cosmetics to an increasing degree. Hair-setting compositions in such gel preparations and also the gel-preparation should satisfy the following requirements. Nontoxic, clear, colorless, nonsticky, good setting action, not very hygroscopic, good consistency.

The copolymers used here also exhibit some properties which are in need of improvement. With the exception of some nonionic copolymers, such as Luviskol K90, K30, Luviskol VA 64 (BASF) or polyvinylformamide, most gels are cloudy to opaque. The readiness to absorb water and also the stickiness of hair treated with these copolymers is likewise too high. The setting effect is likewise also in need of improvement.

The polymers according to the invention do not have the above disadvantages. It has been found that cationizable, preferably N-containing monomers with a content of up to 5% by weight, preferably 2 to 4% by weight, produce particularly clear gels with good setting. Surprisingly, it has been found that a small content of monomer C leads to significantly improved properties.

DE-C 12 61 822 describes copolymers of N-vinylcaprolactam with, for example, N-vinylimidazole and N-vinylpyrrolidone. The copolymers serve as agents for reducing pigment migration during the dyeing of fiber material with pigment dye liquors.

EP 0 455 081 describes copolymers of
35 to 65% by weight of N-vinylcaprolactam,
35 to 65% by weight of a mixture of 5 to 50 parts by weight of N-vinylimidazole and 10 to 60 parts by weight of N-vinylpyrrolidone (which corresponds to a VI:VP (VI/VP) ratio between 1:12 (1/12) and 5:1 (5/1))
0 to 4% by weight of further free-radically copolymerizable monomers,
   and the use of such copolymers as hair-setting and hair care agents.

WO 9831328 describes aqueous preparations comprising (a) 0.1 to 10% by weight of a copolymer based on N-vinylcaprolactam, N-vinylimidazole, N-vinylpyrrolidone and (b) 0.1 to 10% by weight of at least one polyoxyethylene C₆-C₁₅-monoalkyl ether, and their use in cosmetic formulations.

EP 0709411 describes soluble copolymers containing 15 to 84.99% by weight of at least one monomer from the group of N-vinylcaprolactam, N-vinylimidazole, N-vinylpyrrolidone in alcoholic solution.

We have found the use of polymers of
1 to 98.9% by weight of vinylcaprolactam (monomer A)
1 to 98.9% by weight of vinylpyrrolidone (monomer B)
0.1 to 5% by weight of vinylimidazole (monomer C)
0 to 10% by weight of monomer D
0 to 10% by weight (based on the total amount of monomer) of polymer E,
   where the weight ratio of monomer C to monomer B (monomer C/monomer B) is less than 1:12 (1/12),
   preferably
30 to 59% by weight of vinylcaprolactam (monomer A)
40 to 69% by weight of vinylpyrrolidone (monomer B)
1 to 4% by weight of vinylimidazole (monomer C)
0 to 10% by weight of monomer D
0 to 10% by weight (based on the total amount of monomer) of polymer E,
   where the weight ratio of monomer C to monomer B (monomer C/monomer B) is less than 1:13, in hair cosmetics.

Particular preference is given to using polymers of
35 to 50% by weight of vinylcaprolactam (monomer A)
49 to 62% by weight of vinylpyrrolidone (monomer B)
1 to 3% by weight of vinylimidazole (monomer C)
0 to 10% by weight of monomer D
0 to 10% by weight (based on the total amount of monomer) of polymer E,
   where the weight ratio of monomer C to monomer B is less than or equal to 1:14,
   in hair cosmetics, in particular hair-setting compositions.

Particular preference is given to polymers in which the ratio of monomer C to monomer B is less than or equal to 1:15, in particular 1:20, very particularly 1:23.

N-Vinylcaprolactam (monomer A) is understood as meaning N-vinyl-ε-caprolactam. Monomer A is used in an amount of from 1 to 98.9% by weight, preferably 30 to 59% by weight, in particular 35 to 50% by weight.

Vinylpyrrolidone is used as monomer B.
Monomer B is used in an amount of from 1 to 98.9% by weight, preferably 40 to 69% by weight, in particular 49 to 62% by weight.

As monomer C, use is made of a vinylimidazole of the general formula XI, in which R⁴⁰ to R⁴², independently of one another, are hydrogen, C₁-C₄-alkyl or phenyl. Preferably, R⁴⁰ to R⁴² are hydrogen and methyl. 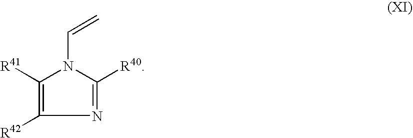
Monomer C is used in an amount of from 0.1 to 5% by weight, preferably 1 to 4% by weight, in particular 1 to 3% by weight.

The following monomers can be used as monomers D:

The preferred additionally used ethylenically unsaturated can be described by the following general formula:
X—C(O)CR²⁰═CHR¹⁹
where
X is chosen from the group of radicals —OH, —OM, —OR²¹, NH₂, —NHR²¹, N(R²¹)₂;
M is a cation chosen from the group consisting of: Na⁺, K⁺, Mg⁺⁺, Ca⁺⁺, Zn⁺⁺, NH₄⁺, alkylammonium, dialkylammonium, trialkylammonium and tetraalkylammonium;
the radicals R²¹ may be identical or different and are chosen from the group consisting of —H, C₁-C₄₀ linear- or branched-chain alkyl radicals, N,N-dimethylaminoethyl, 2-hydroxyethyl, 2-methoxyethyl, 2-ethoxyethyl, hydroxypropyl, methoxypropyl or ethoxypropyl.
R²⁰ and R¹⁹ are chosen independently of one another from the group consisting of: —H, C₁-C₈ linear- or branched-chain alkyl chains, methoxy, ethoxy, 2-hydroxyethoxy, 2-methoxyethoxy and 2-ethoxyethyl.

Representative but nonlimiting examples of suitable monomers (D) are, for example, acrylic acid or methacrylic acid and salts, esters and amides thereof. The salts can be derived from any nontoxic metal, ammonium or substituted ammonium counterions.

The esters can be derived from C₁-C₄₀ linear, C₃-C₄₀ branched-chain or C₃-C₄₀ carbocyclic alcohols, from polyfunctional alcohols having 2 to about 8 hydroxyl groups, such as ethylene glycol, hexylene glycol, glycerol and 1,2,6-hexanetriol, from aminoalcohols or from alcohol ethers, such as methoxyethanol and ethoxyethanol, (alkyl) polyethylene-glycols, (alkyl) polypropylene glycols or ethoxylated fatty alcohols, for example C₁₂-C₂₄-fatty alcohols reacted with 1 to 200 ethylene oxide units.

Also suitable are N,N-dialkylaminoalkyl acrylates and methacrylates and N-dialkylaminoalkylacrylamides and -methacrylamides of the general formula (VII) 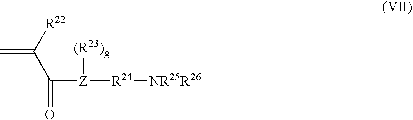
where
R²²=H, alkyl having 1 to 8 carbon atoms,
R²³=H, methyl,
R²⁴=alkylene having 1 to 24 carbon atoms, optionally substituted by alkyl,.
R²⁵, R²⁶=C₁-C₄₀ alkyl radical,
Z=nitrogen when g=1 or oxygen when g=0

The amides may be unsubstituted, N-alkyl or N-alkylamino monosubstituted or N,N-dialkyl-substituted or N,N-dialkylamino-disubstituted, in which the alkyl or alkylamino groups are derived from C₁-C₄₀ linear, C₃-C₄₀ branched-chain or C₃-C₄₀ carbocyclic units. In addition, the alkylamino groups may be quaternized.

Preferred comonomers of the formula VII are N,N-dimethylaminomethyl (meth)acrylate, N,N-diethylaminomethyl (meth)acrylate, N,N-dimethylaminoethyl (meth)acrylate, N,N-diethylaminoethyl (meth)acrylate, N-[3-(dimethylamino)propyl]methacrylamide and N-[3-(dimethylamino)propyl]acrylamide.

Monomers (D) which can likewise be used are substituted acrylic acids, and salts, esters and amides thereof, where the substituents on the carbon atoms are in the two or three position on the acrylic acid, and are chosen independently of one another from the group consisting of C₁-C₄ alkyl, —CN, COOH, particularly preferably methacrylic acid, ethacrylic acid and 3-cyanoacrylic acid. These salts, esters and amides of these substituted acrylic acids can be chosen as described above for the salts, esters and amides of acrylic acid.

Other suitable monomers (D) are allyl esters of C₁-C₄₀ linear, C₃-C₄₀ branched-chain or C₃-C₄₀ carbocyclic carboxylic acids, vinyl or allyl halides, preferably vinyl chloride and allyl chloride, vinyl ethers, preferably methyl, ethyl, butyl or dodecyl vinyl ether, vinyl- or allyl-substituted heterocyclic compounds, preferably vinylpyridine, vinyloxazoline and allylpyridine. Also suitable are N-vinylimidazole derivatives of the general formula VIII, in which R²⁷ to R²⁹, independently of one another, are hydrogen, C₁-C₄-alkyl or phenyl:

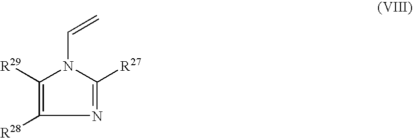

Further suitable monomers (D) are diallylamines of the general formula (IX) 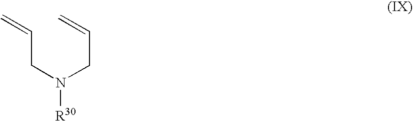
where R³⁰=C₁- to C₂₄-alkyl

Further suitable monomers (D) are vinylidene chloride; and hydrocarbon having at least one carbon-carbon double bond, preferably styrene, alpha-methylstyrene, tert-butylstyrene, butadiene, isoprene, cyclohexadiene, ethylene, propylene, 1-butene, 2-butene, isobutylene, vinyltoluene, and mixtures of these monomers. Particularly suitable comonomers (D) are acrylic acid, methacrylic acid, ethylacrylic acid, methyl acrylate, ethyl acrylate, propyl acrylate, n-butyl acrylate, isobutyl acrylate, t-butyl acrylate, 2-ethylhexyl acrylate, decyl acrylate, methyl methacrylate, ethyl methacrylate, propyl methacrylate, n-butyl methacrylate, isobutyl methacrylate, t-butyl methacrylate, 2-ethylhexyl methacrylate, decyl methacrylate, methyl ethacrylate, ethyl ethacrylate, n-butyl ethacrylate, isobutyl ethacrylate, t-butyl ethacrylate, 2-ethylhexyl ethacrylate, decyl ethacrylate, stearyl (meth)acrylate, 2,3-dihydroxypropyl acrylate, 2,3-dihydroxypropyl methacrylate, 2-hydroxyethyl acrylate, hydroxypropyl acrylates, 2-hydroxyethyl methacrylate, 2-hydroxyethyl ethacrylate, 2-methoxyethyl acrylate, 2-methoxyethyl methacrylate, 2-methoxyethyl ethacrylate, 2-ethoxyethyl methacrylate, 2-ethoxyethyl ethacrylate, hydroxypropyl methacrylates, glyceryl monoacrylate, glyceryl monomethacrylate, polyalkylene glycol (meth)acrylates, unsaturated sulfonic acids, such as, for example, acrylamidopropanesulfonic acid;
acrylamide, methacrylamide, ethacrylamide, N-methylacrylamide, N,N-dimethylacrylamide, N-ethylacrylamide, N-isopropylacrylamide, N-butylacrylamide, N-t-butylacrylamide, N-octylacrylamide, N-t-octylacrylamide, N-octadecylacrylamide, N-phenylacrylamide, N-methylmethacrylamide, N-ethylmethacrylamide, N-dodecylmethacrylamide, 1-vinylimidazole, 1-vinyl-2-methylvinylimidazole, N,N-dimethylaminomethyl (meth)acrylate, N,N-diethylaminomethyl (meth)acrylate, N,N-dimethylaminoethyl (meth)acrylate, N,N-diethylaminoethyl (meth)acrylate, N,N-dimethylaminobutyl (meth)acrylate, N,N-diethylaminobutyl (meth)acrylate, N,N-dimethylaminohexyl (meth)acrylate, N,N-dimethylaminooctyl (meth)acrylate, N,N-dimethylaminododecyl (meth)acrylate, N-[3-(dimethylamino)propyl]methacrylamide, N-[3-(dimethylamino)propyl]acrylamide, N-[3-(dimethylamino)butyl]methacrylamide, N-[8-(dimethylamino)octyl]methacrylamide, N-[12-(dimethylamino)dodecyl]methacrylamide, N-[3-(diethylamino)propyl]methacrylamide, N-[3-(diethylamino)propyl]acrylamide;
malice acid, fumaric acid, maleic anhydride and its half-esters, crotonic acid, itaconic acid, diallyldimethylammonium chloride, vinyl ethers (for example: methyl, ethyl, butyl or dodecyl vinyl ether), methyl vinyl ketone, maleimide, vinylpyridine, vinylimidazole, vinylfuran, styrene, styrene sulfonate, allyl alcohol, and mixtures thereof.
Of these, particular preference is given to acrylic acid, methacrylic acid, maleic acid, fumaric acid, crotonic acid, maleic anhydride and its half-esters, methyl acrylate, methyl methacrylate, ethyl acrylate, ethyl methacrylate, n-butyl acrylate, n-butyl methacrylate, t-butyl acrylate, t-butyl methacrylate, isobutyl acrylate, isobutyl methacrylate, 2-ethylhexyl acrylate, stearyl acrylate, stearyl methacrylate, N-t-butylacrylamide, N-octylacrylamide, 2-hydroxyethyl acrylate, hydroxypropyl acrylates, 2-hydroxyethyl methacrylate, hydroxypropyl methacrylates, alkylene glycol (meth)acrylates, styrene, unsaturated sulfonic acids, such as, for example, acrylamidopropanesulfonic acid, vinyl ethers (e.g. methyl, ethyl, butyl or dodecyl vinyl ether), 1-vinyl-2-methylimidazole, N,N-dimethylaminomethyl methacrylate and N-[3-(dimethylamino)propyl]methacrylamide; 3-methyl-1-vinylimidazolium chloride, 3-methyl-1-vinylimidazolium methyl sulfate, N,N-dimethylaminoethyl methacrylate, N-[3-(dimethylamino)propyl]methacrylamide quaternized with methyl chloride, methyl sulfate or diethyl sulfate.

Monomers with a basic nitrogen atom can be quaternized here in the following way:

Suitable for quaternizing the amines are, for example, alkyl halides having 1 to 24 carbon atoms in the alkyl group, e.g. methyl chloride, methyl bromide, methyl iodide, ethyl chloride, ethyl bromide, propyl chloride, hexyl chloride, dodecyl chloride, lauryl chloride and benzyl halides, in particular benzyl chloride and benzyl bromide. Further suitable quaternizing agents are dialkyl sulfates, in particular dimethyl sulfate or diethyl sulfate. The quaternization of the basic amines can also be carried out with alkylene oxides such as ethylene oxide or propylene oxide, in the presence of acids. Preferred quaternizing agents are: methyl chloride, dimethyl sulfate or diethyl sulfate.

The quaternization can be carried out before the polymerization or after the polymerization.

Furthermore, the reaction products of unsaturated acids, such as, for example, acrylic acid or methacrylic acid, with a quaternized epichlorohydrin of the general formula (X) can be used (R³¹=C₁- to C₄₀-alkyl).

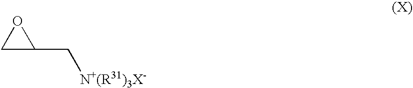

Examples therefore are, for example:
(meth)acryloyloxyhydroxypropyltrimethylammonium chloride and
(meth)acryloyloxyhydroxypropyltriethylammonium chloride.

The basic monomers can also be cationized by neutralizing them with mineral acids, such as, for example, sulfuric acid, hydrochloric acid, hydrobromic acid, hydroiodic acid, phosphoric acid or nitric acid, or with organic acids, such as, for example, formic acid, acetic acid, lactic acid or citric acid.

Further suitable monomers (D) are open-chain N-vinylamide compounds of the general formula (I) 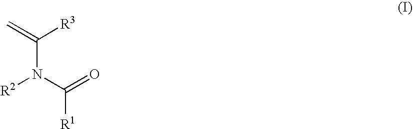
where R¹, R², R³═H or C₁- to C₆-alkyl, and
open-chain N-vinylamide compound, such as, for example, N-vinyl-formamide, N-vinyl-N-methylformamide, N-vinylacetamide, N-vinyl-N-methylacetamide, N-vinyl-N-ethylacetamide, N-vinylpropionamide, N-vinyl-N-methylpropionamide and N-vinylbutyramide. From this group of monomers, preference is given to using N-vinylformamide.

Further suitable monomers (D) are also polyether acrylates, which, for the purpose of this invention, are generally under-stood as meaning esters α,β-ethylenically unsaturated mono- and dicarboxylic acids with polyetherols. Suitable polyetherols are linear or branched substances having terminal hydroxyl groups which contain ether bonds. In general, they have a molecular weight in the range from about 150 to 20 000. Suitable polyetherols are polyalkylene glycols, such as polyethylene glycols, polypropylene glycols, polytetrahydrofurans and alkylene oxide copolymers. Suitable alkylene oxides for the preparation of alkylene oxide copolymers are, for example, ethylene oxide, propylene oxide, epichlorohydrin, 1,2- and 2,3-butylene oxide. The alkylene oxide copolymers can contain the copolymerized alkylene oxide units in randomly distributed form or in the form of blocks. Preference is given to ethylene oxide/propylene oxide copolymers. Preferred monomers D are polyether acrylates of the general formula II 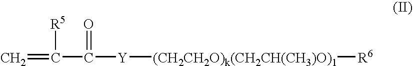
in which

the order of the alkylene oxide units is arbitrary,
k and l, independently of one another, are an integer from 0 to 500, where the sum of k and l is at least 5,
R⁵ is hydrogen or C₁-C₈-alkyl, and
R⁶ is hydrogen or C₁-C₁₈-alkyl,
Y is O or NR⁷, where R⁷ is hydrogen, C₁-C₈-alkyl or C₅-C₈-cycloalkyl.

Preferably, k is an integer from 1 to 500, in particular 3 to 250. Preferably, 1 is an integer from 0 to 100.

Preferably, R⁵ is hydrogen, methyl, ethyl, n-propyl, isopropyl, n-butyl, sec-butyl, tert-butyl, n-pentyl or n-hexyl, in particular hydrogen, methyl or ethyl.

Preferably, R⁶ in the formula II is hydrogen, methyl, ethyl, n-propyl, isopropyl, n-butyl, sec-butyl, n-pentyl, n-hexyl, octyl, 2-ethylhexyl, decyl, lauryl, palmityl or stearyl.

Preferably, Y in the formula II is O or NH.

Suitable polyether acrylates are, for example, the polycondensation products of the abovementioned α,β-ethylenically unsaturated mono- and/or dicarboxylic acids and their acid chlorides, acid amides and anhydrides with polyetherols. Suitable polyetherols can be prepared easily by reacting ethylene oxide, 1,2-propylene oxide and/or epichlorohydrin with a starter molecule such as water or a short-chain alcohol R⁶—OH. The alkylene oxides can be used individually, alternating one after the other or as a mixture. The polyether acrylates can be used on their own or in mixtures for the preparation of the polymers used according to the invention.

Crosslinking monomers (D) which may be used are compounds with at least two ethylenically unsaturated double bonds, such as, for example, esters of ethylenically unsaturated carboxylic acids, such as acrylic acid or methacrylic acid and polyhydric alcohols, ethers of at least dihydric alcohols, such as, for example, vinyl ether or allyl ether.

Examples of the parent alcohols are dihydric alcohols, such as 1,2-ethanediol, 1,2-propanediol, 1,3-propanediol, 1,2-butanediol, 1,3-butanediol, 2,3-butanediol, 1,4-butanediol, but-2-ene-1,4-diol, 1,2-pentanediol, 1,5-pentanediol, 1,2-hexanediol, 1,6-hexanediol, 1,10-decanediol, 1,2-dodecanediol, 1,12-dodecanediol, neopentyl glycol, 3-methylpentane-1,5-diol, 2,5-dimethyl-1,3-hexanediol, 2,2,4-trimethyl-1,3-pentanediol, 1,2-cyclohexanediol, 1,4-cyclohexanediol, 1,4-bis(hydroxymethyl)cyclohexane, neopentyl glycol monohydroxypivalate, 2,2-bis(4-hydroxyphenyl)propane, 2,2-bis[4-(2-hydroxypropyl)phenyl]propane, diethylene glycol, triethylene glycol, tetraethylene glycol, dipropylene glycol, tripropylene glycol, tetrapropylene glycol, 3-thiopentane-1,5-diol, and polyethylene glycols, polypropylene glycols and polytetrahydrofurans with molecular weights of in each case 200 to 10 000. Apart from the homopolymers of ethylene oxide and/or propylene oxide, it is also possible to use block copolymers of ethylene oxide or propylene oxide or copolymers which contain ethylene oxide and propylene oxide groups in incorporated form. Examples of parent alcohols with more than two OH groups are trimethylolpropane, glycerol, pentaerythritol, 1,2,5-pentanetriol, 1,2,6-hexanetriol, triethoxycyanuric acid, sorbitan, sugars, such as sucrose, glucose, mannose. The polyhydric alcohols can of course also be used following reaction with ethylene oxide or propylene oxide as the corresponding ethoxylates or propoxylates, respectively. The polyhydric alcohols can also firstly be converted to the corresponding glycidyl ethers by reaction with epichlorohydrin.

Further suitable crosslinkers are the vinyl esters or the esters of monohydric, unsaturated alcohols with ethylenically unsaturated C₃- to C₆-carboxylic acids, for example acrylic acid, methacrylic acid, itaconic acid, maleic acid or fumaric acid. Examples of such alcohols are allyl alcohol, 1-buten-3-ol, 5-hexen-1-ol, 1-octen-3-ol, 9-decen-1-ol, dicyclopentenyl alcohol, 10-undecen-1-ol, cinnamyl alcohol, citronellol, crotyl alcohol or cis-9-octadecen-1-ol. It is, however, also possible to esterify the monohydric, unsaturated alcohols with polybasic carboxylic acids, for example malonic acid, tartaric acid, trimellitic acid, phthalic acid, terephthalic acid, citric acid or succinic acid.

Further suitable crosslinkers are esters of unsaturated carboxylic acids with the above-described polyhydric alcohols, for example oleic acid, crotonic acid, cinnamic acid or 10-undecenoic acid.

Also suitable are straight-chain or branched, linear or cyclic aliphatic or aromatic hydrocarbons which have at least two double bonds which, in the case of the aliphatic hydrocarbons, must not be conjugated, e.g. divinylbenzene, divinyltoluene, 1,7-octadiene, 1,9-decadiene, 4-vinyl-1-cyclohexene, trivinylcyclohexane or polybutadienes with molecular weights of from 200 to 20 000.

Also suitable are amides of unsaturated carboxylic acids, such as, for example, acrylic and methacrylic acid, itaconic acid, maleic acid, and N-allylamines of at least difunctional amines, such as, for example, 1,2-diaminomethane, 1,2-diaminoethane, 1,3-diaminopropane, 1,4-diaminobutane, 1,6-diaminohexane, 1,12-dodecanediamine, piperazine, diethylenetriamine or isophoronediamine. Likewise suitable are the amides of allylamine and unsaturated carboxylic acids, such as acrylic acid, methacrylic acid, itaconic acid, maleic acid, or at least dibasic carboxylic acids, as have been described above.

In addition, triallylamine or corresponding ammonium salts, e.g. triallylmethylammonium chloride or methyl sulfate, are suitable as crosslinkers.

Furthermore, N-vinyl compounds of urea derivates, at least difunctional amides, cyanurates or urethanes, for example of urea, ethyleneurea, propyleneurea or tartardiamide, e.g. N,N′-divinylethyleneurea or N,N′-divinylpropyleneurea, can be used.

Further suitable crosslinkers are divinyldioxane, tetraallylsilane or tetravinylsilane.

Particularly preferred crosslinkers are, for example, methylenebisacrylamide, divinylbenzene, triallylamine and triallylammonium salts, divinylimidazole, N,N′-divinylethyleneurea, reaction products of polyhydric alcohols with acrylic acid or methacrylic acid, methacrylic esters and acrylic esters of polyalkylene oxides or polyhydric alcohols which have been reacted with ethylene oxide and/or propylene oxide and/or epichlorohydrin, and also allyl or vinyl ethers of polyhydric alcohols, for example 1,2-ethanediol, 1,4-butanediol, diethylene glycol, trimethylolpropane, glycerol, pentaerythritol, sorbitan and sugars, such as sucrose, glucose, mannose.

Very particularly preferred crosslinkers are pentaerythritol triallyl ether, allyl ethers of sugars such as sucrose, glucose and mannose, divinylbenzene, methylenebisacrylamide, N,N′-divinylethyleneurea, and (meth)acrylic esters of glycol, butanediol, trimethylolpropane or glycerol or (meth)acrylic esters of glycol, butanediol, trimethylolpropane or glycerol reacted with ethylene oxide and/or epichlorohydrin.

The content of the monomers (D) is 0 to 10% by weight, preferably 0 to 5% by weight, very particularly preferably 0 to 2% by weight.

The polymer (E) is preferably chosen from
E1) polyether-containing compounds
E2) polymers which contain at least 5% by weight of vinylpyrrolidone units in copolymerized form
E3) polymers which contain at least 50% by weight of vinyl alcohol units
E4) natural substances E4) which contain saccharide structures. As polyether-containing compound El) it is possible to use either polyalkylene oxides based on ethylene oxide, propylene oxide, butylene oxide and other alkylene oxides, or polyglycerol. The polymers contain the following structural units depending on the type of monomer building blocks.
—CH₂)₂-0-, —(CH₂)₃-0-, —(CH₂)₄-0-, —CH₂—CH(R⁹)-0-, —CH₂—CHOR¹⁰—CH₂-0-
   where
R⁹ is C₁-C₂₄-alkyl;
R¹⁰ is hydrogen, C₁-C₂₄-alkyl, R⁹—C(═O)—, R⁹—NH—C(═O)—.

The structural units here may either be homopolymers or random copolymers and block copolymers.

As polymer (E), preference is given to using polymers of the general formula II with a molecular weight >300 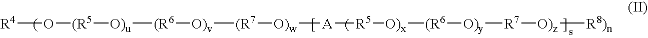
in which the variables, independently of one another, have the following meaning:
R⁴ is hydrogen, C₁-C₂₄-alkyl, R⁹—C(═O)—, R⁹—NH—C(═O)—, polyalcohol radical;
R⁸ is hydrogen, C₁-C₂₄-alkyl, R⁹—C(═O)—, R⁹—NH—C(═O)—;
R⁵ to R⁷
   are —(CH₂)₂—, —(CH₂)₃—, —(CH₂)₄—, —CH₂—CH(R⁹)—, —CH₂—CHOR¹⁰—CH₂—;
R⁹ is C₁-C₂₄-alkyl;
R¹⁰ is hydrogen, C₁-C₂₄-alkyl, R⁹—C(═O)—, R⁹—NH—C(═O)—;
A is —C(═O)—O, —C(═O)—B—C(═O)—O, —C(═O)—NH—BNH—C(═O)—O;
B is —(CH₂)ₜ—, arylene, optionally substituted;
n is 1 to 1000;
s is 0 to 1000;
t is 1 to 12;
u is 1 to 5000;
v is 0 to 5000;
w is 0 to 5000:
x is 0 to 5000;
y is 0 to 5000;
z is 0 to 5000.

The terminal primary hydroxyl groups of the polyethers prepared on the basis of polyalkylene oxides, and the secondary OH groups of polyglycerol may here either be present freely in unprotected form, or else etherified or esterified with alcohols with a chain length of C₁-C₂₄, with carboxylic acids of chain length C₁-C₂₄, respectively, or be reacted with isocyanates to give urethanes.

Alkyl radicals for R⁴ and R⁸ to R¹⁰ which may be mentioned are branched or unbranched C₁-C₂₄-alkyl chains, preferably methyl, ethyl, n-propyl, 1-methylethyl, n-butyl, 1-methylpropyl-, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methyl-pentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methyl-propyl, 1-ethyl-2-methylpropyl, n-heptyl, 2-ethylhexyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl or n-eicosyl.

Preferred representatives of the abovementioned alkyl radicals which may be mentioned are branched or unbranched C₁-C₁₂-, particularly preferably C₁-C₆-alkyl chains.

The molecular weight of the polyethers is in the range greater than 300 (number-average), preferably in the range from 300 to 100 000, particularly preferably in the range from 500 to 50 000, very particularly preferably in the range from 800 to 40 000.

Advantageously, use is made of homopolymers of ethylene oxide or copolymers with an ethylene oxide content of from 40 to 99% by weight. For the ethylene oxide polymers to be used in preference, the content of copolymerized ethylene oxide is thus 40 to 100 mol %. Suitable comonomers for these copolymers are propylene oxide, butylene oxide and/or isobutylene oxide. For example, copolymers of ethylene oxide and propylene oxide, copolymers of ethylene oxide and butylene oxide, and copolymers of ethylene oxide, propylene oxide and at least one butylene oxide are suitable. The ethylene oxide content in the copolymers is preferably 40 to 99 mol %, the propylene oxide content is 1 to 60 mol % and the content of butylene oxide in the copolymers is 1 to 30 mol %. Beside straight-chain homo- or copolymers it is also possible to use branched homo- or copolymers.

Branched polymers can be prepared by adding ethylene oxide and, if appropriate, also propylene oxide and/or butylene oxides onto, for example, polyalcohol radicals, e.g. onto pentaerythritol, glycerol, or onto sugar alcohols, such as D-sorbitol and D-mannitol, but also onto polysaccharides, such as cellulose and starch. The alkylene oxide units may be present in randomly distributed form or in the form of blocks within the polymer.

It is, however, also possible to use polyesters of polyalkylene oxides and aliphatic or aromatic dicarboxylic acids, e.g. oxalic acid, succinic acid, adipic acid and terephthalic acid with molar masses of from 1500 to 25 000, as described, for example, in EP-A-0 743 962, as polyether-containing compound. Furthermore, it is also possible to use polycarbonates by reacting polyalkylene oxides with phosgene or carbonates, such as, for example, diphenyl carbonate, and polyurethanes by reacting polyalkylene oxides with aliphatic and aromatic diisocyanates.

As polyether (E), particular preference is given to polymers of the general formula II with an average molecular weight of from 300 to 100 000 (number-average), in which the variables, independently of one another, have the following meaning:
R⁴ is hydrogen, C₁-C₁₂-alkyl, R⁹—C(═O)—, R⁹—NH—C(═O)—, polyalcohol radical;
R⁸ is hydrogen, C₁-C₁₂-alkyl, R⁹—C(═O)—, R⁹—NH—C(═O)—;
R⁵ to R⁷
   are —(CH₂)₂—, —(CH₂)₃—, —(CH₂)₄—, —CH₂—CH(R⁹)—, —CH₂—CHOR¹⁰—CH₂—;
R⁹ is C₁-C₁₂-alkyl;
R¹⁰ is hydrogen, C₁-C₁₂-alkyl, R⁹—C(═O)—, R⁹—NH—C(═O)—;
n is 1 to 8;
s is 0;
u is 2 to 2000;
v is 0 to 2000;
w is 0 to 2000.

Very particularly preferred polyethers are polymers of the general formula II with an-average molecular weight of from 500 to 50 000 (number-average), in which the variables, independently of one another, have the following meaning:
R⁴ is hydrogen, C₁-C₆-alkyl, R⁹—C(═O)—, R⁹—NH—C(═O)—;
R⁸ is hydrogen, C₁-C₆-alkyl, R⁹—C(═O)—, R⁹—NH—C(═O)—;
R⁵ to R⁷
   are —(CH₂)₂—, —(CH₂)₃—, —(CH₂)₄—, —CH₂—CH(R⁹)—, —CH₂—CHOR¹⁰—CH₂—;
R⁹ is C₁-C₆-alkyl;
R¹⁰ is hydrogen, C₁-C₆-alkyl, R⁹—C(═O)—, R⁹—NH—C(═O)—;
n is 1;
s is 0;
u is 5 to 500;
v is 0 to 500;
w is 0 to 500.

In addition, polyethers (E1) which may be used are also homopolymers and copolymers of polyalkylene oxide-containing ethylenically unsaturated monomers, such as, for example, polyalkylene oxide (meth)acrylates, polyalkylene oxide vinyl ethers, polyalkylene oxide (meth)acrylamides, polyalkylene oxide allyamides or polyalkylene oxide vinylamides. It is of course also possible to use copolymers of such monomers with other ethylenically unsaturated monomers.

Polyether-containing compounds (El) which may be used are, however, also reaction products of polyethyleneimines with alkylene oxides. In this case, the alkylene oxides used are preferably ethylene oxide, propylene oxide, butylene oxide and mixtures thereof, particularly preferably ethylene oxide. The polyethyleneimines used may be polymers with number-average molecular weights of from 300 to 20 000, preferably 500 to 10 000, very particularly preferably 500 to 5000. The weight ratio between alkylene oxide used and polyethyleneimine is in the range from 100:1 to 0.1:1, preferably in the range 50:1 to 0.5:1, very particularly preferably in the range 20:1 to 0.5:1.

However, polymers E2) which contain at least 5% by weight of vinylpyrrolidone units can also be used as polymer (E). These polymers preferably contain a vinylpyrrolidone content of at least 10% by weight, very particularly preferably of at least 30% by weight.

Suitable vinylpyrrolidone comonomers for the synthesis of the polymers (E2) are, for example, N-vinylcaprolactam, N-vinylimidazole, N-vinyl-2-methylimidazole, N-vinyl-4-methylimidazole, 3-methyl-1-vinylimidazolium chloride, 3-methyl-1-vinylimidazolium methyl sulfate, diallylammonium chloride, styrene, alkylstyrenes.

Further suitable comonomers for the preparation of the polymers (E3) are, for example, monoethylenically unsaturated c₃-C₆-carboxylic acids, such as, for example, acrylic acid, methacrylic acid, crotonic acid, fumaric acid, and their esters, amides and nitriles, such as, for example, methyl acrylate, ethyl acrylate, methyl methacrylate, ethyl methacrylate, stearyl methacrylate, hydroxyethyl acrylate, hydroxypropyl acrylate, hydroxybutyl acrylate, hydroxyethyl methacrylate, hydroxypropyl methacrylate, hydroxyisobutyl acrylate, hydroxyisobutyl methacrylate, monomethyl maleate, dimethyl maleate, monoethyl maleate, diethyl maleate, 2-ethylhexyl acrylate, 2-ethylhexyl methacrylate, maleic anhydride, and half-esters thereof, alkylene glycol (meth)acrylates, acrylamide, methacrylamide, N-dimethylacrylamide, N-tert-butylacrylamide, acrylonitrile, methacrylonitrile, vinyl ethers, such as, for example, methyl, ethyl, butyl or dodecyl vinyl ether, cationic monomers, such as dialkylaminoalkyl (meth)acrylates and dialkylaminoalkyl (meth)acrylamides, such as dimethylaminothyl acrylate, diethylaminoethyl acrylate, diethylaminoethyl methacrylate, and the salts of the last-mentioned monomers with carboxylic acids or mineral acids, and the quaternized products.

The polymers (E) are prepared by known processes, for example solution, precipitation, suspension or emulsion polymerization using compounds which form free radicals under the polymerization conditions. The polymerization temperatures are usually in the range from 30 to 200° C., preferably 40 to 110° C. Suitable initiators are, for example, azo and peroxy compounds, and the customary redox initiator systems, such as combinations of hydrogen peroxide and reducing compounds, for example sodium sulfite, sodium bisulfite, sodium formaldehyde sulfoxylate and hydrazin. These systems can, if appropriate, additionally also comprise small amounts of a heavy metal salt.

The homopolymers and copolymers (polymers E2) have K values of at least 7, preferably 10 to 250. However, the polymers can have K values up to 300. The K values are determined in accordance with H. Fikentscher, Cellulose-Chemie, Volume 13, 58 to 64 and 71 to 74 (1932) in aqueous solution at 25° C, at concentrations which, depending on the K value range, are between 0.1% and 5%.

Polymers (E3) which have at least 50% by weight of vinyl alcohol units can, however, also as polymer (E). Preferably, these polymers comprises at least 70% by weight, very particularly preferably 80% by weight, of polyvinyl alcohol units. Such polymers are usually prepared by polymerization of a vinyl ester and subsequent at least partial alcoholysis, aminolysis or hydrolysis. Preference is given to vinyl esters of linear and branched C₁-C₁₂-carboxylic acids, and vinyl acetate is very particularly preferred. The vinyl esters can of course also be used in a mixture.

Suitable vinyl ester comonomers for the synthesis of the polymers (E3) are, for example, N-vinylcaprolactam, N-vinylpyrrolidone, N-vinylimidazole, N-vinyl-2-methylimidazole, N-vinyl-4-methylimidazole, 3-methyl-1-vinylimidazolium chloride, 3-methyl-1-vinylimidazolium methyl sulfate, diallylammonium chloride, styrene, alkylstyrenes.

Further suitable comonomers for the preparation of the polymers (E3) are, for example, monoethylenically unsaturated c₃-C₆-carboxylic acids, such as, for example, acrylic acid, methacrylic acid, crotonic acid, fumaric acid, and their esters, amides and nitriles, such as, for example, methyl acrylate, ethyl acrylate, methyl methacrylate, ethyl methacrylate, stearyl methacrylate, hydroxyethyl acrylate, hydroxypropyl acrylate, hydroxybutyl acrylate, hydroxyethyl methacrylate, hydroxypropyl methacrylate, hydroxyisobutyl acrylate, hydroxyisobutyl methacrylate, monomethyl maleate, dimethyl maleate, monoethyl maleate, diethyl maleate, 2-ethylhexyl acrylate, 2-ethylhexyl methacrylate, maleic anhydride, and half-esters thereof, alkylene glycol (meth)acrylates, acrylamide, methacrylamide, N-dimethylacrylamide, N-tert-butylacrylamide, acrylonitrile, methacrylonitrile, vinyl ethers, such as, for example, methyl, ethyl, butyl or dodecyl vinyl ether, cationic monomers, such as dialkylaminoalkyl (meth)acrylates and dialkylaminoalkyl (meth)acrylamides, such as dimethylaminoethyl acrylate, diethylaminoethyl acrylate, diethylaminoethyl methacrylate, and the salts of the last-mentioned monomers with carboxylic acids or mineral acids, and the quaternized products.

Preferably, polymers (E3) are polymers which are prepared by homopolymerization of vinyl acetate and subsequent at least partial hydrolysis, alcoholysis or aminolysis.

The polymers (E3) are prepared by known processes, for example solution, precipitation, suspension or emulsion polymerization using compounds which form free radicals under the polymerization conditions. The polymerization temperatures are usually in the range from 30 to 200° C., preferably 40 to 110° C. Suitable initiators are, for example, azo and peroxy compounds, and the customary redox initiator systems, such as combinations of hydrogen peroxide and reducing compounds, for example sodium sulfite, sodium bisulfite, sodium formaldehyde sulfoxylate and hydrazin. These systems can, if appropriate, additionally also comprise small amounts of a heavy metal salt.

To prepare the polymers (E3), the ester groups of the original monomers and, if appropriate, further monomers are at least partially cleaved after the polymerization by hydrolysis, alcoholysis or aminolysis. In the text below, this process step is generally referred to as saponification. The saponification takes place in a manner known per se by adding a base or acid, preferably by adding a sodium hydroxide or potassium hydroxide solution in water and/or alcohol. Particular preference is given to using methanolic sodium hydroxide or potassium hydroxide solutions. The saponification is carried out at temperatures in the range from 10 to 80° C., preferably in the range from 20 to 60° C. The degree of saponification depends on the amount of base or acid used, on the saponification temperature, the saponification time and the water content of the solution.

Particularly preferred polymers (E3) are polymers which are prepared by homopolymerization of vinyl acetate and subsequent at least partial saponification. Such polymers containing polyvinyl alcohol units are available under the name Mowiol®. As polymer (E) it is also possible, however, to use natural substances (E4) which contain saccharide structures. Such natural substances are, for example, saccharides of vegetable or animal origin or products which are formed by metabolization by microorganisms, and degradation products thereof. Suitable polymers (E4) are, for example, oligosaccharides, polysaccharides, oxidatively, enzymatically or hydrolytically degraded polysaccharides, oxidatively hydrolytically degraded or oxidatively enzymatically degraded polysaccharides, chemically modified oligosaccharides or polysaccharides and mixtures thereof.

Preferred products are the compounds specified in U.S. Pat. No. 5,334,287 at column 4, line 20 to column 5, line 45.

As monomers E and D, preference is given to using water-soluble or water-dispersible monomers, preferably water-soluble monomers. Water-soluble is understood as meaning that the monomers are soluble in water at 25° C. in an amount of at least 2% by weight.

The polymers are prepared by free-radical polymerization of the monomers A to D, if appropriate in the presence of the polymers E. The process is carried out under the customary polymerization conditions, for example by the methods of precipitation, suspension, emulsion, solution or dispersion polymerization, and also bulk polymerization. Solution polymerization in water or an organic solvent, usually an alcohol or in a water/alcohol mixture has proven to be particularly advantageous. The process here is usually carried out at temperatures of from 60 to 130° C., it being possible to carry out the reaction at atmospheric pressure, autogenous pressure or reduced pressure.

Initiators which may be used for the free-radical polymerization are the water-soluble and water-insoluble peroxo and/or azo compounds customary for this purpose, for example alkali metal or ammonium peroxydisulfates, dibenzoyl peroxide, tert-butyl perpivalate, tert-butyl per-2-ethylhexanoate, di-tert-butyl peroxide, tert-butyl hydroperoxide, azobisisobutyronitrile, azobis(2-amidinopropane) dihydrochloride or 2,2′-azobis(2-methylbutyronitrile). Also suitable are initiator mixtures or redox initiator systems, such as, for example, ascorbic acid/iron(II) sulfate/sodium peroxodisulfate, tert-butyl hydroperoxide/sodium disulfite, tert-butyl hydroperoxide/sodium hydroxymethanesulfinate. The initiators can be used in the customary amounts, for example 0.05 to 5% by weight, based on the amount of monomers to be polymerized.

The K values of the polymers are in a range between 10 to 350, preferably 20 to 200 and particularly preferably 35 to 110, very particularly between 40 and 80. The K values are measured in accordance with Fikentscher, Cellulosechemie, Vol. 13, pp. 58-64 (1932) at 25° C. as 1% strength in aqueous solution.

The amount of monomers and solvent is advantageously chosen such that 20 to 80% strength by weight solutions of the copolymers are obtained. The polymer E is initially introduced in dissolved, swollen or dispersed form in water prior to the addition of monomer in the reaction vessel, preference being given to using a 3 to 70% strength by weight, in particular 3 to 50% strength by weight mixture.

The polymer mixture can, if appropriate, be subjected to an additional after-polymerization and, if appropriate to an after-treatment by steam distillation, treatment with acids/alkalis or oxidizing or reducing agents. In a preferred embodiment, the polymer is subjected to steam distillation.

For the stabilization, the polymer solution is treated with Euxyl® K 100 (Schülke & Mayr), with Phenonip® (Clariant) or with an alternative stabilizer.

Pulverulent products can be obtained by precipitation, spray-drying from suitable solvent systems or freeze-drying.

The gels formulated with the film formers described are characterized by improved properties compared with the prior art.

The polymers according to the invention can advantageously be used in cosmetic preparations, in particular hair cosmetic preparations.

The term cosmetic preparations is to be understood in the wide sense and means all those preparations which are suitable for application to skin and/or hair and/or nails and pursue a purpose other than a purely medicinal-therapeutic one.

The polymers according to the invention can be used in skin cosmetic preparations.

For example, the polymers according to the invention are used in cosmetic compositions for cleansing the skin. Such cosmetic cleansing compositions are chosen from bar soaps, such as toilet soaps, curd soaps, transparent soaps, luxury soaps, deodorant soaps, cream soaps, baby soaps, skin protection soaps, abrasive soaps and syndets, liquid soaps, such as pasty soaps, soft soaps and washing pastes, and liquid washing, showering and bathing preparations, such as washing lotions, shower baths and shower gels, foam baths, oil baths and scrub preparations.

Preferably, the polymers according to the invention are used in cosmetic compositions for the care and protection of the skin, in nail care compositions, and in preparations for decorative cosmetics.

Use in skincare compositions, intimate care compositions, foot care compositions, deodorants, light protection compositions, repellents, shaving compositions, hair removal compositions, antiacne compositions, make-up, mascara, lipsticks, eye shadows, kohl pencils, eyeliners, blushers, powders and eyebrow pencils is particularly preferred.

The skincare compositions are in particular in the form of W/O or OW skin creams, day and night creams, eye creams, face creams, antiwrinkle creams, moisturizing creams, bleaching creams, vitamin creams, skin lotions, care lotions and moisturizing lotions.

In the cosmetic preparations, the polymers according to the invention can develop particular effects. The polymers can, inter alia, contribute to the moisturization and conditioning of the skin and to an improvement in the feel of the skin. The polymers can also act as thickeners in the formulations. By adding the polymers according to the invention, a considerable improvement in the skin compatibility can be achieved in certain formulations.

The copolymers according to the invention are present in the skin cosmetic preparations in an amount of from about 0.001 to 20% by weight, preferably 0.01 to 10% by weight, very particularly preferably 0.1 to 5% by weight, based on the total weight of the composition.

Depending on the field of use, the compositions according to the invention can be applied in a form suitable for skin care, such as, for example, in the form of a cream, foam, gel, stick, powder, mousse, milk or lotion.

Besides the polymers according to the invention and suitable solvents, the skin cosmetic preparations can also comprise additives customary in cosmetics, such as emulsifiers, preservatives, perfume oils, cosmetic active ingredients, such as phytantriol, vitamin A, E and C, retinol, bisabolol, panthenol, light protection agents, bleaches, colorants, tinting agents, tanning agents (e.g. dihydroxyacetone), collagen, protein hydrolysates, stabilizers, pH regulators, dyes, salts, thickeners, gel formers, bodying agents, silicones, humectants, refatting agents and further customary additives.

Suitable solvents are, in particular, water and lower monoalcohols or polyols with 1 to 6 carbon atoms or mixtures thereof; preferred monoalcohols or polyols are ethanol, isopropanol, propylene glycol, glycerol and sorbitol.

Further customary additives which may be present are fatty bodies, such as mineral and synthetic oils, such as, for example, paraffins, silicone oils and aliphatic hydrocarbons having more than 8 carbon atoms, animal and vegetable oils, such as, for example, sunflower oil, coconut oil, avocado oil, olive oil, lanolin, or waxes, fatty acids, fatty acid esters, such as, for example, triglycerides of C₆-C₃₀-fatty acids, wax esters, such as, for example, jojoba oil, fatty alcohols, Vaseline, hydrogenated lanolin and acetylated lanolin. It is of course also possible to use mixtures of these.

Customary thickeners in such formulations are crosslinked polyacrylic acids and derivatives thereof, polysaccharides, such as xanthan gum, agar agar, alginates or tyloses, carboxymethylcellulose or hydroxycarboxymethylcellulose, fatty alcohols, monoglycerides and fatty acids, polyvinyl alcohol and polyvinylpyrrolidone.

The polymers according to the invention can also be mixed with conventional polymers if specific properties are to be set.

Suitable conventional polymers are, for example, anionic, cationic, amphoteric and neutral polymers.

Examples of anionic polymers are homopolymers and copolymers of acrylic acid and methacrylic acid or salts thereof, copolymers of acrylic acid and acrylamide and salts thereof; sodium salts of polyhydroxycarboxylic acids, water-soluble or water-dispersible polyesters, polyurethanes and polyureas. Particularly suitable polymers are copolymers of t-butyl acrylate, ethyl acrylate, methacrylic acid (e.g. Luvimer® 100P), copolymers of ethyl acrylate and methacrylic acid (e.g. Luvimer® MAE), copolymers of N-tert-butylacrylamide, ethyl acrylate, acrylic acid (Ultrahold® 8, strong), copolymers of vinyl acetate, crotonic acid and, if appropriate, further vinyl esters (e.g. Luviset® grades), maleic anhydride copolymers, optionally reacted with alcohols, anionic polysiloxanes, e.g. carboxyfunctional, copolymers of vinylpyrrolidone, t-butyl acrylate, methacrylic acid (e.g. Luviskol® VBM), copolymers of acrylic acid and methacrylic acid with hydrophobic monomers, such as, for example, C₄-C₃₀-alkyl esters of meth(acrylic acid), C₄-C₃₀-alkyl vinyl esters, C₄-C₃₀-alkyl vinyl ethers and hyaluronic acid.

Other suitable polymers are cationic polymers with the INCI name Polyquaternium, e.g. copolymers of vinylpyrrolidone/N-vinylimidazolium salts (Luviquat® FC, Luviquat® HM, Luviquat® MS, Luviquat® Care), copolymers of N-vinylpyrrolidone/dimethylaminoethyl methacrylate, quaternized with diethyl sulfate (Luviquat® PQ 11), copolymers of N-vinylcaprolactam/N-vinylpyrrolidone/N-vinylimidazolium salts (Luviquat® Hold); cationic cellulose derivatives (Polyquaternium-4 and -10), acrylamide copolymers (Polyquaternium-7) and chitosan.

Suitable further polymers are also neutral polymers, such as polyvinylpyrrolidones, copolymers of N-vinylpyrrolidone and vinyl acetate and/or vinyl propionate, polysiloxanes, polyvinylcaprolactam and copolymers containing N-vinylpyrrolidone, polyethyleneimines and salts thereof, polyvinylamines and salts thereof, cellulose derivatives, polyaspartic acid salts and derivatives.

To establish certain properties, the preparations may additionally also comprise conditioning substances based on silicone compounds. Suitable silicone compounds are, for example, polyalkylsiloxanes, polyarylsiloxanes, polyarylalkylsiloxanes, polyether siloxanes or silicone resins.

The copolymers according to the invention are used in cosmetic preparations, the preparation of which takes place in accordance with the customary principles familiar to the person skilled in the art.

Such formulations are advantageously in the form of emulsions, preferably in the form of water-in-oil (W/O) or oil-in-water (O/W) emulsions. However, according to the invention, it is also possible and, if appropriate, advantageous to choose other types of formulation, for example hydrodispersions, gels, oils, oleogels, multiple emulsions, for example in the form of W/O/W or O/W/O emulsions, anhydrous ointments or ointment bases etc.

Emulsions which can be used according to the invention are prepared by known methods.

Besides the copolymer according to the invention, the emulsions comprise customary constituents, such as fatty alcohols, fatty acid esters and in particular fatty acid triglycerides, fatty acids, lanolin and derivatives thereof, natural or synthetic oils or waxes and emulsifiers in the presence of water.

The selection of additives specific to the type of emulsion, and the preparation of suitable emulsions is described, for example, in Schrader, Grundlagen und Rezepturen der Kosmetika [Fundamentals and formulations of cosmetics], Hüthig Buch verlag, Heidelberg, 2nd Edition, 1989, third part, which is hereby expressly incorporated by reference.

For example, a skin cream which can be used according to the invention may be in the form, for example, of a W/O emulsion. Such an emulsion comprises an aqueous phase which is emulsified in an oil or fatty phase by means of a suitable emulsifier system.

The concentration of the emulsifier system in this type of emulsion is about 4 and 35% by weight, based on the total weight of the emulsion; the fatty phase constitutes about 20 and 60% by weight and the aqueous phase about 20 and 70% by weight, in each case based on the total weight of the emulsion. The emulsifiers are those which are customarily used in this type of emulsion. They are chosen, for example, from: C₁₂-C₁₈-sorbitan fatty acid esters; esters of hydroxystearic acid and C₁₂-C₃₀-fatty alcohols; monoesters and diesters of C₁₂-C₁₈-fatty acids and glycerol or polyglycerol; condensates of ethylene oxide and propylene glycols; oxypropylenated/oxyethylenated C₁₂-C₂₀-fatty alcohols; polycyclic alcohols, such as sterols; aliphatic alcohols with a high molecular weight, such as lanolin; mixtures of oxypropylenated/polyglycerylated alcohols and magnesium isostearate; succinate esters of polyoxyethylenated or polyoxypropylenated fatty alcohols; and mixtures of magnesium lanolate, calcium lanolate, lithium lanolate, zinc lanolate or aluminum lanolate and hydrogenated lanolin or lanolin alcohol.

Suitable fatty components which may be present in the fatty phase of the emulsions include hydrocarbon oils, such as paraffin oil, purcellin oil, perhydrosqualene and solutions of microcrystalline waxes in these oils; animal or vegetable oils, such as sweet almond oil, avocado oil, calophylum oil, lanolin and derivatives thereof, castor oil, sesame oil, olive oil, jojoba oil, karite oil, hoplostethus oil; mineral oils whose distillation start under atmospheric pressure is about 250° C. and whose distillation end point is 410° C., such as, for example, Vaseline oil; esters of saturated and unsaturated fatty acids, such as alkyl myristates, e.g. isopropyl, butyl or cetyl myristate, hexadecyl stearate, ethyl or isopropyl palmitate, octanoic acid or decanoic acid triglycerides and cetyl ricinoleate.

The fatty phase may also comprise silicone oils which are soluble in other oils, such as dimethylpolysiloxane, methylphenylpolysiloxane and the silicone-glycol copolymer, fatty acids and fatty alcohols.

In order to favor the retention of oils, it is also possible to use waxes, such as, for example, carnauba wax, candellila wax, beeswax, microcrystalline wax, ozokerite wax and Ca, Mg and Al oleates, myristates, linoleates and stearates.

In general, these water-in-oil emulsions are prepared by adding the fatty phase and the emulsifier to a mixing container. The container is heated at a temperature of from 70 to 750° C., then the oil-soluble ingredients are added, and water which has been heated beforehand to the same temperature and into which the water-soluble ingredients have been dissolved beforehand is added with stirring; the mixture is stirred until an emulsion of the desired fineness is obtained, which is then left to cool to room temperature, if appropriate with stirring to a lesser extent.

In addition, a care emulsion according to the invention can be in the form of an O/W emulsion. Such an emulsion usually comprises an oil phase, emulsifiers which stabilize the oil phase in the water phase, and an aqueous phase, which is usually in thickened form.

The aqueous phase of the O/W emulsion of the preparations according to the invention comprises, if appropriate,
alcohols, diols or polyols, and ethers thereof, preferably ethanol, isopropanol, propylene glycol, glycerol, ethylene glycol monoethyl ether;
customary thickeners or gel formers, such as, for example, crosslinked polyacrylic acids and derivatives thereof, polysaccharides, such as xanthan gum or alginates, carboxymethylcellulose or hydroxycarboxymethylcellulose, fatty alcohols, polyvinyl alcohol and polyvinylpyrrolidone.

The oil phase comprises oil components customary in cosmetics, such as, for example:
esters of saturated and/or unsaturated, branched and/or unbranched C₃-C₃₀-alkanecarboxylic acids and saturated and/or unsaturated, branched and/or unbranched C₃-C₃₀-alcohols, of aromatic carboxylic acids and saturated and/or unsaturated, branched and/or unbranched C₃-C₃₀-alcohols, for example isopropyl myristate, isopropyl stearate, hexyldecyl stearate, oleyl oleate; and also synthetic, semisynthetic and natural mixtures of such esters, such as jojoba oil;
branched and/or unbranched hydrocarbons and hydrocarbon waxes;
silicone oils, such as cyclomethicone, dimethylpolysiloxane, diethylpolysiloxane, octamethylcyclotetrasiloxane, and mixtures thereof;
dialkyl ethers;
mineral oils and mineral waxes;
triglycerides of saturated and/or unsaturated, branched and/or unbranched C₈-C₂₄-alkanecarboxylic acids; they can be chosen from synthetic, semisynthetic or natural oils, such as olive oil, palm oil, almond oil or mixtures.

Suitable emulsifiers are preferably o/W emulsifiers, such as polyglycerol esters, sorbitan esters or partially esterified glycerides.

The preparation can take place by melting the oil phase at about 80° C.; the water-soluble constituents are dissolved in hot water, and added to the oil phase slowly and with stirring; the mixture is homogenized and stirred until cold.

The polymers according to the invention are also suitable for use in washing and shower gel formulations, and also bathing preparations.

Besides the polymers according to the invention, such formulations usually comprise anionic surfactants as base surfactants, and amphoteric and nonionic surfactants as cosurfactants, and also lipids, perfume oils, dyes, organic acids, preservatives and antioxidants, and also thickeners/gel formers, skin conditioners and moisturizers.

In the washing, showering and bathing preparations it is possible to use all of the anionic, neutral, amphoteric or cationic surfactants customarily used in body-cleansing compositions.

The formulations comprise 2 to 50% by weight of surfactants, preferably 5 to 40% by weight, particularly preferably 8 to 30% by weight.

Suitable anionic surfactants are, for example, alkyl sulfates, alkyl ether sulfates, alkylsulfonates, alkylarylsulfonates, alkyl succinates, alkyl sulfosuccinates, N-alkoyl sarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

For example, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate, triethanolamine dodecylbenzenesulfonate are suitable.

Suitable amphoteric surfactants are, for example, alkylbetaines, alkylamidopropylbetaines, alkylsulfobetaines, alkyl glycinates, alkylcarboxyglycinates, alkyl amphoacetates or amphopropionates, alkyl amphodiacetates or amphodipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 moles per mol of alcohol. Also suitable are alkylamine oxides, mono- or dialkylalkanolamides, fatty acid esters of polyethylene glycols, ethoxylated fatty acid amides, alkyl polyglycosides or sorbitan ether esters.

Furthermore, the washing, showering and bathing preparations can comprise customary cationic surfactants, such as, for example, quaternary ammonium compounds, for example cetyltrimethylammonium chloride.

In addition, further customary cationic polymers may also be used, thus, for example, copolymers of acrylamide and dimethyldiallylammonium chloride (Polyquaternium-7), cationic cellulose derivatives (Polyquaternium-4, -10), guar hydroxypropyltrimethylammonium chloride (INCI: Hydroxypropyl Guar Hydroxypropyltrimonium Chloride), copolymers of N-vinylpyrrolidone and quaternized N-vinylimidazole (Polyquaternium-16, -44, -46), copolymers of N-vinypyrrolidone/dimethylaminoethyl methacrylate, quaternized with diethyl sulfate (Polyquaternium-11) and others.

In addition, the washing and shower gel formulations and bathing preparations can comprise thickeners, such as, for example, sodium chloride, PEG-55, propylene glycol oleate, PEG-120 methyl glucose dioleate and others, and also preservatives, further active ingredients and auxiliaries and water.

Hair cosmetic preparations comprise, in particular, styling agents and/or conditioning agents in hair cosmetic preparations such as hair treatments, hair mousses, (hair) gels or hairsprays, hair lotions, hair rinses, hair shampoos, hair emulsions, end fluids, neutralizers for permanent waves, hair colorants and bleaches, “hot-oil treatment” preparations, conditioners, setting lotions or hairsprays. Depending on the field of use, the hair cosmetic preparations can be applied in the form of an (aerosol) spray, (aerosol) foam, gel, gel spray, cream, lotion or wax.

The hair cosmetic formulations according to the invention comprise, in a preferred embodiment,
a) 0.05 to 20% by weight of the polymer according to the invention
b) 20 to 99.95% by weight of water and/or alcohol
c) 0 to 79.5% by weight of further constituents

Alcohol is understood as meaning all alcohols customary in cosmetics, e.g. ethanol, isopropanol, n-propanol.

Further constituents are understood as meaning the additives customary in cosmetics, for example propellants, antifoams, interface-active compounds, i.e. surfactants, emulsifiers, foam formers and solubilizers. The interface-active compounds used may be anionic, cationic, amphoteric or neutral. Further customary constituents may also be, for example, preservatives, perfume oils, opacifiers, active ingredients, UV filters, care substances, such as panthenol, collagen, vitamins, protein hydrolysates, alpha-and beta-hydroxycarboxylic acids, protein hydrolysates, stabilizers, pH regulators, dyes, viscosity regulators, gel formers, dyes, salts, humectants, refatting agents, complexing agents and further customary additives.

Gel formers which can be used are all gel formers customary in cosmetics. These include slightly crosslinked polyacrylic acid, for example Carbomer (INCI) or Acrylates/C10-30 Alkyl Acrylate Crosspolymer (INCI), Acrylates/Beheneth-25 Methacrylate Copolymer (INCI), PVM/MA decadiene crosspolymer, cellulose derivatives, e.g. hydroxypropylcellulose, hydroxyethylcellulose, cationically modified celluloses, polysaccharides, e.g. xanthum gum, hydroxypropyl starch phosphate, potato starch modified, caprylic/capric triglyceride, sodium acrylates copolymer, Polyquaternium-32 (and) Paraffinum Liquidum (INCI), Sodium Acrylates Copolymer (and) Paraffinum Liquidum (and) PPG-1 Trideceth-6, acrylamidopropyltrimonium chloride/acrylamide copolymer, steareth-10 allyl ether acrylates copolymer, Polyquaternium-37 (and) Paraffinum Liquidum (and) PPG-1 Trideceth-6, polyacrylamides and C13-14 isoparaffin and laureth-7, C13-14 isoparaffin and mineral oil and sodium polyacrylate and polyacrylamide and polysorbate 85, C13-14 isoparaffin and isostearyl isostearate and sodium polyacrylate and polyacrylamide and polysorbate 60, acrylates/aminoacrylates/C10-30 alkyl PEG-20 itaconate copolymer, acrylates/steareth-20 itaconate copolymer, acrylates/ceteth-20 itaconate copolymer, polyquaternium 37 (and) propylene glycol dicaprate dicaprylate (and) PPG-1 trideceth-6, polyquaternium-7, polyquaternium-44.

Also included here are all styling and conditioner polymers known in cosmetics which can be used in combination with the polymers according to the invention if very specific properties are to be set.

Suitable conventional hair cosmetic polymers are, for example, anionic polymers. Such anionic polymers are homopolymers and copolymers of acrylic acid and methacrylic acid or salts thereof, copolymers of acrylic acid and acrylamide and salts thereof; sodium salts of polyhydroxycarboxylic acids, water-soluble or water-dispersible polyesters, polyurethanes (Luviset® P.U.R.) and polyureas. Particularly suitable polymers are copolymers of t-butyl acrylate, ethyl acrylate, methacrylic acid (e.g. Luvimer® 100P), copolymers of N-tert-butylacrylamide, ethyl acrylate, acrylic acid (Ultrahold® 8, Strong), copolymers of vinyl acetate, crotonic acid, and, if appropriate, further vinyl esters (e.g. Luviset® grades), maleic anhydride copolymers, optionally reacted with alcohols, anionic polysiloxanes, e.g. carboxyfunctional, copolymers of vinylpyrrolidone, t-butyl acrylate, methacrylic acid (e.g. Luviskol® VBM).

In addition, the group of the polymers suitable for combining with the polymers according to the invention comprises, for example, Balancer CR (National Starch; acrylate copolymer), Balancer 0/55 (National Starch; acrylate copolymer), Balancer 47 (National Starch; octylacrylamide/acrylate/butylaminoethyl methacrylate copolymer), Aquaflex® FX 64 (ISP; isobutylene/ethylmaleimide/hydroxyethylmaleimide copolymer), Aquaflex® SF-40 (ISP/National Starch; VP/vinyl caprolactam/DMAPA acrylate copolymer), Allianz® LT-120 (ISP/Rohm & Haas; acrylate/C1-2 succinate/hydroxyacrylate copolymer), Aquarez® HS (Eastman; polyester-1), Diaformer® Z-400 (Clariant; methacryloylethylbetaine/methacrylate copolymer), Diaformer® Z-711 (Clariant; methacryloylethyl N-oxide/methacrylate copolymer), Diaformer® Z-712 (Clariant; methacryloylethyl N-oxide/methacrylate copolymer), Omnirez® 2000 (ISP; monoethylester of poly(methyl vinyl ether/maleic acid in ethanol)), Amphomer® HC (National Starch; acrylate/octylacrylamide copolymer), Amphomer® 28-4910 (National Starch; octylacrylamide/acrylate/butylaminoethyl methacrylate copolymer), Advantage® HC 37 (ISP; terpolymer of vinylcaprolactam/vinylpyrrolidone/dimethylaminoethyl methacrylate), Acudyner 258 (Rohm & Haas; acrylate/hydroxy ester acrylate copolymer), Luviset® PUR (BASF, polyurethane-1), Luviflex® Silk (BASF), Eastman® AQ48 (Eastman).

Very particularly preferred anionic polymers are acrylates with an acid number greater than or equal to 120 and copolymers of t-butyl acrylate, ethyl acrylate, methacrylic acid.

Further suitable hair cosmetic polymers are-cationic polymers with the INCI name Polyquaternium, e.g. copolymers of vinylpyrrolidone/N-vinylimidazolium salts (Luviquat® FC, Luviquat® HM, Luviquat® MS, Luviquat® Care), copolymers of N-vinylpyrrolidone/dimethylaminoethyl methacrylate, quaternized with diethyl sulfate (Luviquat® PQ 11), copolymers of N-vinylcaprolactam N-vinylpyrrolidone/N-vinylimidazolium salts (Luviquat® Hold); cationic cellulose derivatives (Polyquaternium-4 and -10), acrylamide copolymers (Polyquaternium-7).

In addition, cationic guar derivatives, such as Guar Hydroxypropyltrimonium Chloride (INCI) may be used.

Suitable further hair cosmetic polymers are also neutral polymers, such as polyvinylpyrrolidones, copolymers of N-vinylpyrrolidone and vinyl acetate and/or vinyl propionate, polysiloxanes, polyvinylcaprolactam and copolymers containing N-vinylpyrrolidone, polyethyleneimines and salts thereof, polyvinylamines and salts thereof, cellulose derivatives, polyaspartic acid salts and derivatives.

To establish certain properties, the preparations may additionally also comprise conditioning substances based on silicone compounds. Suitable silicone compounds are, for example, polyalkylsiloxanes, polyarylsiloxanes, polyarylalkylsiloxanes, polyether siloxanes, silicone resins or dimethicone copolyols (CTFA) and aminofunctional silicone compounds, such as amodimethicones (CTFA).

The polymers according to the invention are particularly suitable as setting agents in hairstyling preparations, in particular hairsprays (aerosol sprays and pump sprays without propellant gas) and hair mousses (aerosol mousses and pump mousses without propellant gas).

### EXAMPLES

The polymers were prepared by the customary methods of free-radial polymerization in water. The synthesis of the polymers I, II, III, IV may be described below by way of representation for all other preparation procedures.
I: Copolymer of monomer A, B, C polymerized in the presence of polymer E
   Preparation of a polymer of 56.5% by weight of N-vinylpyrrolidone, 40% by weight of vinylcaprolactam and 3.5% by weight of vinylimidazole in the presence of 5% by weight (based on the total amount of monomer) of Mowiol® 4-88.
   A solution of 10 g of Mowiol® 4-88 in 50 g of water is initially introduced and heated to 75° C. A solution of 113 g of vinylpyrrolidone, 7 g of vinylimidazole and 80 g of vinylcaprolactam in 300 g of water is added over the course of 3 hours and, starting at the same time as the monomer addition, a solution of 1 g of Wako V 50 in 100 g of water is added over the course of 4 hours. The mixture is then after-polymerized for 2 h at an internal temperature of 750° C.
II: Copolymer of monomer A, B, C, D
   Preparation of a polymer of 55.0% by weight of N-vinylpyrrolidone, 40% by weight of vinylcaprolactam, 2.5% by weight of vinylimidazole and 2.5% by weight of Bisomer® Sl0W (Laport).
   200 g of water is initially introduced and heated to 75° C. A solution of 110 g of vinylpyrrolidone, 5 g of vinylimidazole, 5 g of Bisomer SLOW and 80 g of vinylcaprolactam in 195 g of water is added over the course of 2 hours and, starting at the same time as the monomer addition, a solution of 1 g of Wako V 50 in 20 g of water is added over the course of 2.5 hours. The mixture is then after-polymerized for 1 h at an internal temperature of 75° C.
III: Copolymer of monomer A, B, C, D polymerized in the presence of polymer E
   Preparation of a polymer of 55% by weight of N-vinylpyrrolidone, 35% by weight of vinylcaprolactam, 2% by weight of vinylimidazole and 8% of vinylformamide in the presence of 5% by weight of Pluriole® E 4000 (based on the total amount of monomer).
   10 g of Pluriol® E 4000 is initially introduced into 200 g of water and heated to 75° C. A solution of 110 g of vinylpyrrolidone, 4 g of vinylimidazole, 16 g of vinylformamide and 70 g of vinylcaprolactam in 195 g of water is added over the course of 2 hours, and, starting at the same time as the monomer addition, a solution of 1 g of Wako V 50 in 20 g of water is added over the course of 2.5 hours. The mixture is then after-polymerized for 1 h at an internal temperature of 75° C.
IV: Copolymer of monomer A, B, C
   Preparation of a polymer of 51.5% by weight of N-vinylpyrrolidone, 45% by weight of vinylcaprolactam and 3.5% by weight of vinylimidazole.
   50 g of water is initially introduced and heated to 75° C. A solution of 103 g of vinylpyrrolidone, 7 g of vinylimidazole and 90 g of vinylcaprolactam in 300 g of water is added over the course of 3 hours and, starting at the same time as the monomer addition, a solution of 1 g of Wako V 50 in 100 g of water is added over the course of 4 hours. The mixture is then after-polymerized for 2 h at an internal temperature of 75° C.

The gel formulations were assessed according to the following criteria:

Appearance:

The clarity of the gels was assessed relative to known standards

| | | |
|---|---|---|
| 1 | clear | comparable with Luviskol K 30 |
| 2 | almost clear | comparable with Luviskol K 90 |
| 3 | slightly cloudy | poorer than Luviskol K 90 |
| 4 | cloudy | |
| 5 | milky | |

Stickiness:

The stickiness was determined in accordance with Kempf at 75 and 90% relative atmospheric humidity at ambient temperature directly on dried films of the gel formulation.
1 not sticky
2 slightly sticky
3 moderately sticky
4 very sticky
   Flexural Rigidity:

The flexural rigidity was determined on tresses of hair treated with gel. As
1>180 cN
2>150 cN
3>120 cN
4>90 cN
5>60 cN
   Curl Retention from Solution:

The curl retention was determined on tresses of hair treated with a 3% aqueous polymer solution (water).
1>50%
2>40%
3>30%
4>20%
   Curl Retention from the Gel:

The curl retention was determined on tresses of hair treated with gel.
1>80%
2>70%
3>60%
4>50%

### Comparative examples

| | | Stickiness | Stickiness | | CR[%] soln. | |
|---|---|---|---|---|---|---|
| | | (Kempf) | (Kempf) | | 3% aq. | CR[%] gel |
| | | 25° C., | 25° C., | Flexural | 25° C. | 25° C. |
| | | 75% rel. | 90% rel | rigidity | 90% rel. | 90% rel. |
| Composition | Appearance | humidity | humidity | [cN] | humidity | humidity |
| Luvitec VPC | 3 | — | — | — | — | — |
| Luviskol Plus | 4 | — | — | — | — | — |
| Luviskol K30 | 1 | 2 | 3 | 5 | 4 | 4 |
| Luviskol K90 | 2 | 3 | 3 | 2 | 4 | 3 |
| Luviskol VA 64 | 2 | 1 | 5 | 5 | 4 | 4 |

Luvitec VPC: Copolymer of VP/VCap 1:1
Luviskol Plus: Homopolymer of VCap
Luviskol K30: Homopolymer of VP
Luvsikol K90: Homopolymer of VP
Luviskol VA 64: Copolymer of VP/Vac 3:2

### Examples 1

VP-VCap-VI copolymer systems

| | | | | | | | CR[%] | |
|---|---|---|---|---|---|---|---|---|
| | | | | Stickiness | Stickiness | | soln. 3% | |
| | | | | (Kempf) | (Kempf) | | aq. | CR[%] |
| | | | | 25° C., | 25° C., | | 25° C. | 25° C. |
| | | | | 75% rel. | 90% rel. | Flexural | 90% rel. | 90% rel. |
| | Composition | Ratio | Appearance | humidity | humidity | rigidity [cN] | humidity | humidity |
| a) | VP/VI/VCap | 60/10/30 | 4-5 | — | — | — | — | — |
| b) | VP/VI/VCap | 37/3/60 | 4-5 | — | — | — | — | — |
| c) | VP/VCap | 60/40 | 1 | 1 | 1-2 | 4 | 3 | 1 |
| d) | VP/VI/VCap | 65/5/30 | 4 | 0 | 2 | 1 | 2 | 1 |
| e) | VP/VI/VCap | 50/5/45 | 4 | 0 | 1-2 | 1 | 2 | 1 |
| f) | VP/VI/VCap | 56.5/3.5/40 | 2 | 1 | 3 | 1 | 3 | 1 |
| g) | VP/VI/VCap | 57/3/40 | 1 | 1 | 2 | 1 | 3 | 1 |
| h) | VP/VI/VCap | 57.5/2.5/40 | 1 | 1 | 2 | 1 | 3 | 1 |
| i) | VP/VI/VCap | 62.5/2.5/35 | 1 | 1 | 1-2 | 1 | 4 | 2 |
| j) | VP/VI/VCap | 58.5/1.5/40 | 1 | 1 | 1-2 | 2 | 3 | 1 |
| k) | VP/VI/VCap | 52.5/2.5/45 | 1 | 1 | 1-2 | 1 | 3 | 1 |
| l) | VP/VI/VCap | 53/2/45 | 1 | 1 | 1-2 | 1 | 3 | 1 |
| m) | VP/VI/VCap | 51.5/3.5/45 | 2-3 | 1 | 2 | 1 | 3 | 1 |

### Examples 2

VP-VCap-VI copolymer systems comprising a further monomer D (Ex. 2d) or polymerized in the presence of a polymer E (Ex. 2a)+2b)) or comprising further monomer D and polymerized in the presence of a polymer E (Ex. 2c)

| | | | | | | | CR[%] | |
|---|---|---|---|---|---|---|---|---|
| | | | | Stickiness | Stickiness | | soln. 3% | |
| | | | | (Kempf) | (Kempf) | | aq. | CR[%] |
| | | | | 25° C., | 25° C., | | 25° C. | 25° C. |
| | | | | 75% rel. | 90% rel. | Flexural | 90% rel. | 90% rel. |
| | Composition | Ratio | Appearance | humidity | humidity | rigidity [cN] | humidity | humidity |
| a) | VP/VI/VCap + | 56.5/3.5/40 | 1-2 | 1 | 1-2 | 1 | 3 | |
| | Mowiol 4-88 | 5% by wt. | | | | | | |
| b) | VP/VI/VCap + | 60/2.5/37.5 | 1 | 1 | 2 | 1 | 3 | |
| | Tylose H 4000 G | 5% by wt. | | | | | | |
| C) | VP/VI/VCap/VFA + | 55/2/35/8 | 1-2 | 1-2 | 3 | 2 | 3 | |
| | Pluriol E 4000 | 5% by wt. | | | | | | |
| d) | VP/VI/VCap/Bi- | 55/2.5/40/2.5 | 1 | 2 | 4 | 1 | 3 | |
| | somer S10W | | | | | | | |

Application
Preparation procedures:

Carbopol gel (200 g)
3% polymer in 0.5% Carbopol (940 or Ultrez 21)
Batch a): 98.68 g of Carbopol stock solution (1% strength preserved with Euxyl K 100)
   1.32 g of triethanolamine in 250 ml beaker

The TEA is incorporated into the stock solution using a stirrer (about 90 rpm) until the mixture is clear (about 15 min)
Batch b) 6.00 g of polymer (solid)
   ad 100 g of water dist. in 250 ml Erlenmeyer

If batch b) is completely dissolved, it is slowly incorporated into batch a) using a dropping funnel (about 1 drop/sec) at the same stirrer speed. If, after adding the solution dropwise, the finished gel is formed, it is after-stirred for about a further 30 minutes.

Shampoo

Preparation:

Dissolve all components in water, adjust pH and then add thickener.

Aerosol Spray

Preparation:

Weigh in all components. Adjust the pH, and bottle clear solution in pressurized container with propellant gas.

Wax

Preparation:

Weigh in the components of the phase, melt and stir uniformly.

Aerosol mousse

Preparation:

Mix perfume oil phase. Add the components of the aqueous-ethanolic phase one after the other and mix. If stated, add thickener and stir until uniformly distributed. Adjust the pH. Bottle in a pressurized container with propellant gas.

Pump Spray

Preparation:

Stir aqueous phase. Add the components of the ethanolic phase one after the other and distribute uniformly. Then bottle everything in a pump spray bottle.

Pump Mousse

Preparation:

Prepare a uniform mixture from the components and bottle in a pump mousse bottle.

Emulsions of the O/W Type (Hair Rinses, etc.)

Preparation:

Mix oil phase with emulsifiers (if necessary at elevated temperature) and add aqueous phase (with possible thickener, if necessary at elevated temperature) with stirring and homogenize.

Formulations:

Hair gel containing polymer as in Ex. 1g) to 11) and Luviskol K30

| % | Raw material | Supplier | INCI |
|---|---|---|---|
| 0.50 | Carbopol 940 | (6) | Carbomer |
| 87.60 | Water, demin. | | Aqua dem. |
| 0.70 | Triethanolamine Care | (1) | Triethanolamine |
| 6.00 | Polymer 1g) to 11) | (1) | |
| 5.00 | Luviskol K30 solution | (1) | PVP |
| q.s. | Perfume oil | | |
| q.s. | Cremophor RH 40 | (1) | PEG-40 Hydrogenated |
| | | | Castor Oil |
| 0.10 | Euxyl K100 | (42) | Benzyl Alcohol, Methyl- |
| | | | chloroisothiazolinone, |
| | | | Methylisothiazolinone |
| 0.10 | Vitamin E acetate | | Tocopheryl Acetate |

Suppliers
(1) BASF Aktiengesellschaft
(6) B. F. Goodrich Company Chemical Division
(42) Schülke & Mayr GmbH

Hair gel containing polymer as in Ex. 1g) to 11) and Luviskol VA64

| % | Raw material | Supplier | INCI |
|---|---|---|---|
| 0.50 | Carbopol 980 | (6) | Carbomer |
| 87.60 | Water demin. | | Aqua dem. |
| 0.90 | Neutrol TE | (1) | Tetrahydroxypropyl |
| | | | Ethylenediamine |
| 7.00 | Polymer 1g) to 11) | (1) | |
| 4.00 | Luviskol VA64 W | (1) | VP/VA Copolymer |
| q.s. | Perfume oil | | |
| q.s. | Cremophor CO 40 | (1) | PEG-40 Hydrogenated |
| | | | Castor Oil |
| 0.10 | Euxyl K100 | (42) | Benzyl Alcohol, Methyl- |
| | | | chloroisothiazolinone, |
| | | | Methylisothiazolinone |
| 0.10 | 1,2 Propylene glycol Care | (1) | Propylene Glycol |

Suppliers
(1) BASF Aktiengesellschaft
(6) B. F. Goodrich Company Chemical Division
(42) Schülke & Mayr GmbH

Hair gel containing polymer as in Ex. 1g) to 11) and Luviskol K90

| % | Raw material | Supplier | INCI |
|---|---|---|---|
| 0.50 | Carbopol ETD 2001 | (6) | Carbomer |
| 87.60 | Water demin. | | Aqua dem. |
| 0.70 | Triethanolamine Care | (1) | Triethanolamine |
| 6.00 | Polymer 1g) to 11) | (1) | |
| 5.00 | Luviskol K90 | (1) | PVP |
| q.s. | Perfume oil | | |
| q.s. | Cremophor CO 40 | (1) | PEG-40 Hydrogenated |
| | | | Castor Oil |
| 0.10 | Nipagin M | (34) | Methylparaben |
| 0.10 | Isopropylmyristate | (27) | Isopropyl Myristate |

Suppliers
(1) BASF Aktiengesellschaft
(6) B. F. Goodrich Company Chemical Division
(34) Nipa Laboratories Ltd.
(27) Cognis Deutschland GmbH

Hair gel containing polymer as in Ex. 1g) to 11) and Luviquat Hold

| % | Raw material | Supplier | INCI |
|---|---|---|---|
| 10.00 | Polymer 1g) to 11) | (1) | |
| 2.50 | Luviquat Hold | (1) | Polyquaternium-46 |
| 15.00 | Ethanol 96% | | Alcohol |
| 70.30 | Water demin. | | Aqua dem |
| 5.00 | Luviskol K90 | (1) | PVP |
| 0.10 | Perfume oil | | |
| 0.10 | Glycerol | (20) | Glycerin |
| 2.00 | Natrosol 250 HR | (4) | Hydroxyethylcellulose |

Suppliers
(1) BASF Aktiengesellschaft
(6) B. F. Goodrich Company Chemical Division
(20) Merck KGaA
(4) Aqualon GmbH

Hair gel containing polymer as in Ex. 1g) to 11) and Amaze

| % | Raw material | Supplier | INCI |
|---|---|---|---|
| 6.00 | Polymer 1g) to 11) | (1) | |
| 2.00 | Amaze | (72) | Corn Starch Modified |
| 0.50 | Hydagen HCMF | (27) | Chitosan |
| q.s. | Perfume oil | | |
| q.s. | Cremophor CO 40 | (1) | PEG-40 Hydrogenated |
| | | | Castor Oil |
| 0.10 | Abil 8843 | (44) | PEG-14 Dimethicone |
| 0.10 | Euxyl K100 | (42) | Benzyl Alcohol, Methyl- |
| | | | chloroisothiazolinone, |
| | | | Methylisothiazolinone |
| 91.40 | Water demin. | | Aqua dem. |

Suppliers
(1) BASF Aktiengesellschaft
(6) B. F. Goodrich Company Chemical Division
(27) Cognis Deutschland GmbH
(42) Schülke & Mayr GmbH
(44) Th. Goldschmidt AG
(72) National Starch & Chemical Limited

Hair gel containing polymer as in Ex. 1g) to 11) and Styleze CC-10

| % | Raw material | Supplier | INCI |
|---|---|---|---|
| 8.00 | Polymer 1g) to 11) | (1) | |
| 5.00 | Styleze CC-10 | (65) | VP/DMAPA Acrylates |
| | | | Copolymer |
| 0.05 | AMP | (56) | Aminomethyl Propanol |
| 84.85 | Water demin. | | Aqua dem |
| q.s. | Perfume oil | | |
| q.s. | Cremophor RH 40 | (1) | PEG-40 Hydrogenated |
| | | | Castor Oil |
| 0.10 | Dow Corning 190 | (16) | Dimethicone Copolyol |
| 0.10 | Euxyl K100 | (42) | |
| 2.00 | Klucel | (4) | Hydroxypropylcellulose |

Suppliers
(1) BASF Aktiengesellschaft
(4) Aqualon GmbH
(16) Dow Corning Corporation
(42) Schülke & Mayr GmbH
(56) Angus Chemical Company
(65) ISP Global Technologies Deutschland GmbH

Hair gel containing polymer as in Ex. 1g) to 11) and Styleze 2000

| % | Raw material | Supplier | INCI |
|---|---|---|---|
| 6.00 | Polymer 1g) to 11) | (1) | |
| 1.00 | Styleze 2000 | (65) | VP/Acrylates/Lauryl |
| | | | Methacrylate Copolymer |
| 0.26 | AMP | (56) | Aminomethyl Propanol |
| 90.64 | Water demin. | | Aqua dem |
| q.s. | Perfume oil | | |
| q.s. | Cremophor RH 40 | (1) | PEG-40 Hydrogenated |
| | | | Castor Oil |
| 0.10 | Karion F Liquid | (20) | Sorbitol |
| 0.10 | Euxyl K100 | (42) | Benzyl Alcohol, Methyl- |
| | | | chloroisothiazolinone, |
| | | | Methylisothiazolinone |
| 2.00 | Hydroxypropyl guar | — | Hydroxypropylguar |

Suppliers
(1) BASF Aktiengesellschaft
(20) Merck KGaA
(42) Schülke & Mayr GmbH
(56) Angus Chemical Company
(65) ISP Global Technologies Deutschland GmbH

Hair gel containing polymer as in Ex. 1g) to 11) and Allianz LT-120

| % | Raw material | Supplier | INCI |
|---|---|---|---|
| 0.50 | Ultrez 10 | (6) | Carbomer |
| 90.01 | Water demin. | | Aqua dem. |
| 0.70 | Triethanolamine Care | (1) | Triethanolamine |
| 6.00 | Polymer 1g) to 11) | (1) | |
| 2.00 | Allianz LT-120 | (61) | Acrylates/C1-2 |
| | | | Succinates/Hydroxy- |
| | | | acrylates Copolymer |
| 0.19 | AMP | (56) | Aminomethyl Propanol |
| q.s. | Perfume oil | | |
| q.s. | Cremophor CO 40 | (1) | PEG-40 Hydrogenated |
| | | | Castor Oil |
| 0.10 | Pluracare E400 | (1) | PEG-8 |
| 0.10 | Euxyl K100 | (42) | |
| 0.50 | Natrosol 250 HR | (4) | Hydroxyethylcellulose |

Suppliers
(1) BASF Aktiengesellschaft
(4) Aqualon GmbH
(6) B. F. Goodrich Company Chemical Division
(42) Schülke & Mayr GmbH
(56) Angus Chemical Company
(61) Röhm & Haas GmbH

Hair gel containing polymer as in Ex. 1g) to 11) and Fixomer A30

| % | Raw material | Supplier | INCI |
|---|---|---|---|
| 7.00 | Polymer 1g) to 11) | (1) | |
| 7.00 | Fixomer A30 | — | |
| 0.70 | Triethanolamine Care | (1) | Triethanolamine |
| q.s. | Perfume oil | | |
| q.s. | Cremophor CO 40 | (1) | PEG-40 Hydrogenated |
| | | | Castor Oil |
| 0.10 | D-Panthenol USP | (1) | Panthenol |
| 0.10 | Euxyl K100 | (42) | Benzyl Alcohol, Methyl- |
| | | | chloroisothiazolinone, |
| | | | Methylisothiazolinone |
| 84.90 | Water demin. | | Aqua dem. |
| 1.00 | Sepigel 305 | (175) | |

Suppliers
(1) BASF Aktiengesellschaft
(42) Schülke & Mayr GmbH
(175) Seppic

Hair gel containing polymer as in Ex. 1g) to 11) and PVF

| % | Raw material | Supplier | INCI |
|---|---|---|---|
| 0.50 | Carbopol 940 | (6) | Carbomer |
| 90.50 | Water demin. | | Aqua dem. |
| 0.70 | Triethanolamine Care | (1) | Triethanolamine |
| 7.00 | Polymer 1g) to 1l) | (1) | |
| 1.00 | PVF | (72) | Polyvinylformamide |
| q.s. | Perfume oil | | |
| q.s. | Cremophor RH 40 | (1) | PEG-40 Hydrogenated |
| | | | Castor Oil |
| 0.10 | Euxyl K100 | (42) | Benzyl Alcohol, Methyl- |
| | | | chloroisothiazolinone, |
| | | | Methylisothiazolinone |
| 0.10 | Uvinul MC 80 | (1) | Ethylhexyl Methoxy- |
| | | | cinnamate |
| 0.10 | Abil 8843 | (44) | PEG-14 Dimethicone |

Suppliers
(1) BASF Aktiengesellschaft
(6) B. F. Goodrich Company Chemical Division
(42) Schülke & Mayr GmbH
(44) Th. Goldschmidt AG
(72) National Starch & Chemical Limited

Hair gel containing polymer as in Ex. 1g) to 11)

| % | Raw material | Supplier | INCI |
|---|---|---|---|
| 0.50 | Carbopol 940 | (6) | Carbomer |
| 88.50 | Water demin. | | Aqua dem. |
| 0.70 | Triethanolamine Care | (1) | Triethanolamine |
| | | | Ethylenediamine |
| 10.00 | Polymer 1g) to 1l) | (1) | |
| q.s. | Perfume oil | | |
| q.s. | Cremophor CO 40 | (1) | PEG-40 Hydrogenated |
| | | | Castor Oil |
| 0.10 | Euxyl K100 | (42) | Benzyl Alcohol, Methyl- |
| | | | chloroisothiazolinone, |
| | | | Methylisothiazolinone |
| 0.10 | 1,2 Propylene glycol Care | (1) | Propylene Glycol |
| 0.10 | Isopropyl myristate | (27) | Isopropyl Myristate |

Suppliers
(1) BASF Aktiengesellschaft
(6) B. F. Goodrich Company Chemical Division
(27) Cognis Deutschland GmbH
(42) Schülke & Mayr GmbH

Hair gel containing polymer as in Ex. 1g) to 11)

| % | Raw material | Supplier | INCI |
|---|---|---|---|
| 10.00 | Polymer 1g) to 1l) | (1) | |
| 15.00 | Ethanol 96% | | |
| 72.70 | Water demin. | | Aqua dem |
| 0.10 | Perfume oil | | |
| 0.10 | Glycerol | (20) | Glycerin |
| 0.10 | D-Panthenol USP | (1) | Panthenol |
| 2.00 | Natrosol 250 HR | (4) | Hydroxyethylcellulose |

Suppliers
(1) BASF Aktiengesellschaft
(6) B. F. Goodrich Company Chemical Division
(20) Merck KGaA
(4) Aqualon GmbH

Hair gel containing polymer as in Ex. 1g) to 11)

| % | Raw material | Supplier | INCI |
|---|---|---|---|
| 0.50 | Carbopol ETD 2001 | (6) | Carbomer |
| 88.50 | Water demin. | | Aqua dem. |
| 0.70 | Triethanolamine Care | (1) | Triethanolamine |
| 10.00 | Polymer 1g) to 1l) | (1) | |
| q.s. | Perfume oil | | |
| q.s. | Cremophor CO 40 | (1) | PEG-40 Hydrogenated |
| | | | Castor Oil |
| 0.10 | Nipagin M | (34) | Methylparaben |
| 0.10 | Uvinul MC 80 | (1) | Ethylhexyl Methoxy- |
| | | | cinnamate |
| 0.10 | Abil 8843 | (44) | PEG-14 Dimethicone |

Suppliers
(1) BASF Aktiengesellschaft
(6) B. F. Goodrich Company Chemical Division
(34) Nipa Laboratories Ltd.
(44) Th. Goldschmidt AG

Hair gel containing polymer as in Ex. 1g) to 11)

| % | Raw material | Supplier | INCI |
|---|---|---|---|
| 10.00 | Polymer 1g) to 1l) | (1) | |
| q.s. | Perfume oil | | |
| q.s. | Cremophor CO 40 | (1) | PEG-40 Hydrogenated |
| | | | Castor Oil |
| 0.10 | Palatinol A | (1) | Diethyl Phthalate |
| 0.10 | Luvitol EHO | (1) | Cetearyl Ethylhexanoate |
| 0.10 | Cetiol HE | (27) | PEG-7 Glyceryl Cocoate |
| 0.10 | Euxyl K100 | (42) | Benzyl Alcohol, Methyl- |
| | | | chloroisothiazolinone, |
| | | | Methylisothiazolinone |
| 87.70 | Water demin. | | Aqua dem. |
| 2.00 | Luvigel EM | (1) | Caprylic/Capric Tri- |
| | | | glyceryde, Acrylates |
| | | | Copolymer |

Suppliers
(1) BASF Aktiengesellschaft
(27) Cognis Deutschland GmbH
(42) Schülke & Mayr GmbH

Setting solution containing polymer as in Ex. 1g) to 11)

| % | Raw material | Supplier | INCI |
|---|---|---|---|
| 62.60 | Ethanol 96% | | Alcohol |
| 30.00 | Water demin. | | Aqua dem. |
| 0.10 | Dow Corning 190 polyether | (16) | Dimethicone Copolyol |
| 0.10 | Perfume oil | | |
| 0.10 | Uvinul MC 80 | (1) | Ethylhexyl, Methoxy- |
| | | | cinnamate |
| 0.10 | D-Panthenol USP | (1) | Panthenol |
| 7.00 | Polymer 1g) to 1l) | (1) | |

Suppliers
(1) BASF Aktiengesellschaft
(16) Dow Corning Corporation

Setting solution containing polymer as in Ex. 1g) to 11)

| % | Raw material | Supplier | INCI |
|---|---|---|---|
| 0.10 | Dow Corning 190 polyether | (16) | Dimethicone Copolyol |
| 0.05 | Dow Corning 344 fluid | (16) | Cyclomethicone |
| q.s. | Perfume oil | | |
| 53.85 | Ethanol 96% | | Alcohol |
| 40.00 | Water demin. | | Aqua dem. |
| 6.00 | Polymer 1g) to 1l) | (1) | |

Suppliers
(1) BASF Aktiengesellschaft
(16) Dow Corning Corporation

Setting solution containing polymer as in Ex. 1g) to 11)

| % | Raw material | Supplier | INCI |
|---|---|---|---|
| 0.10 | D-Panthenol USP | (1) | Panthenol |
| 0.10 | Nutrilan Keratin W | | |
| 0.10 | Elastin PG 2000 | | Hydrolyzed Elastin |
| 0.40 | Uvinul M 40 | (1) | Benzophenone-3 |
| 10.00 | Water demin. | | Aqua dem. |
| 84.30 | Ethanol 96% | | Alcohol |
| q.s. | Perfume oil | | |
| 5.00 | Polymer 1g) to 1l) | (1) | |

Suppliers
(1) BASF Aktiengesellschaft

Setting solution containing polymer as in Ex. 1g) to 11) and Luviquat FC 550

| % | Raw material | Supplier | INCI |
|---|---|---|---|
| 4.00 | Polymer 1g) to 1l) | (1) | Polyvinylcaprolactam |
| 3.50 | Luviquat FC 550 | (1) | Polyquaternium-16 |
| 72.20 | Ethanol 96% | | Alcohol |
| 20.00 | Water demin. | | Aqua dem. |
| q.s. | Perfume oil | | |

Suppliers
(1) BASF Aktiengesellschaft

Setting solution containing polymer as in Ex. 1g) to 11)

| % | Raw material | Supplier | INCI |
|---|---|---|---|
| 4.00 | Polymer 1g) to 1l) | (1) | |
| 0.20 | Pluracare E 400 | (1) | PEG-8 |
| 0.10 | Perfume oil | | |
| 10.00 | Water demin. | | |
| 85.70 | Ethanol 96% | | Alcohol |

Suppliers
(1) BASF Aktiengesellschaft

Pump spray containing polymer as in Ex. 1g) to 11)

| % | Raw material | Supplier | INCI |
|---|---|---|---|
| 26.00 | Polymer 1g) to 11) | (1) | |
| 73.70 | Ethanol 96% | | Alcohol |
| 0.10 | Perfume oil | | |
| 0.10 | Uvinul MC 80 | (1) | Ethylhexyl Methoxy- |
| | | | cinnamate |
| 0.10 | Dow Corning 190 | (16) | PEG/PPG-18/18 Dimethi- |
| | | | cone |

Suppliers
(1) BASF Aktiengesellschaft
(16) Dow Corning Corporation

Pump spray containing polymer as in Ex. 1g) to 11)

| % | Raw material | Supplier | INCI |
|---|---|---|---|
| 26.00 | Polymer 1g) to 11) | (1) | |
| 4.00 | Luviskol Plus | (1) | Polyvinylcaprolactam |
| 69.60 | Ethanol 96% | | Alcohol |
| 0.10 | Uvinul MC 80 | (1) | Ethylhexyl Methoxy- |
| | | | cinnamate |
| 0.10 | Dow Corning 344 | (16) | Cyclomethicone |
| 0.10 | Dow Corning 556 | (16) | Phenyl Trimethicone |

Suppliers
(1) BASF Aktiengesellschaft
(16) Dow Corning Corporation

Aerosol spray NON VOC containing polymer as in Ex. 1g) to 11)

| % | Raw material | Supplier | INCI |
|---|---|---|---|
| 13.00 | Polymer 1g) to 11) | (1) | |
| 0.10 | Perfume oil | | |
| 0.10 | 1,2 Propylene glycol Care | (1) | Propylene Glykol |
| 0.10 | Citroflex 2 | (53) | Triethyl Citrate |
| 46.70 | Water demin. | | Aqua dem. |
| 40.00 | HFC 152A | — | Hydrofluorocarbon 152a |

Suppliers
(1) BASF Aktiengesellschaft
(53) Pfizer Chemie

Aerosol spray NON VOC containing polymer as in Ex. 1g) to 11) and Luviset CAN

| % | Raw material | Supplier | INCI |
|---|---|---|---|
| 10.00 | Polymer 1g) to 11) | (1) | |
| 2.00 | Luviset CAN | (1) | VA/Crotonates/Vinyl |
| | | | Neodecanoate Copolymer |
| 0.16 | AMP | (56) | Aminomethyl Propanol |
| 0.10 | Perfume oil | | |
| 0.10 | Phytantriol | (1) | Phytantriol |
| 52.64 | Water demin. | | Aqua dem. |
| 35.00 | HFC 152A | — | Hydrofluorocarbon 152a |

Suppliers
(1) BASF Aktiengesellschaft
(56) Angus Chemical Company

Aerosol spray VOC 55 containing polymer as in Ex. 1g) to 11) and Luviset P.U.R.

| % | Raw material | Supplier | INCI |
|---|---|---|---|
| 7.00 | Polymer 1g) to 11) | (1) | |
| 7.00 | Luviset P.U.R. | (1) | Polyurethane-1 |
| | | | Neodecanoate Copolymer |
| 14.30 | Ethanol absolute | | Alcohol |
| 36.50 | Water demin. | | Aqua dem. |
| 0.10 | 1,2 Propylene glycol Care | (1) | Propylene Glycol |
| 0.10 | Perfume oil | | |
| 40.00 | DME | — | Dimethylether |

Suppliers
(1) BASF Aktiengesellschaft

Aerosol spray VOC 55 containing polymer as in Ex. 1g) to 11) and Luviskol Plus

| % | Raw material | Supplier | INCI |
|---|---|---|---|
| 10.00 | Polymer 1g) to 11) | (1) | |
| 5.00 | Luviskol Plus. | (1) | Polyvinylcaprolactam |
| 17.00 | Ethanol absolute | | Alcohol |
| 32.80 | Water demin. | | Aqua dem. |
| 0.10 | Niacinamide | — | Niacinamide |
| 0.10 | Perfume oil | | |
| 35.00 | DME | — | Dimethylether |

Suppliers
(1) BASF Aktiengesellschaft

Aerosol spray VOC 80 containing polymer as in Ex. 1g) to 11) and Luvimer 100P

| % | Raw material | Supplier | INCI |
|---|---|---|---|
| 10.00 | Polymer 1g) to 11) | (1) | |
| 1.00 | Luvimer 100P | (1) | Acrylates Copolymer |
| 0.24 | AMP | (56) | Aminomethyl Propanol |
| 35.00 | Ethanol absolute | | Alcohol |
| 8.56 | Water demin. | | Aqua dem. |
| 0.10 | Belsil CM040 | (156) | Cyclopentasiloxane |
| 0.10 | Perfume oil | | |
| 10.00 | n-Butane | — | Butane |
| 35.00 | DME | — | Dimethylether |

Suppliers
(1) BASF Aktiengesellschaft
(56) Angus Chemical Company
(156) Wacker Chemie GmbH

Aerosol spray VOC 80 containing polymer as in Ex. 1g) to 11) and Luviskol VA37

| % | Raw material | Supplier | INCI |
|---|---|---|---|
| 10.00 | Polymer 1g) to 11) | (1) | |
| 4.00 | Luviskol VA37 | (1) | VP/VA Copolymer |
| 38.00 | Ethanol absolute | | Alcohol |
| 7.70 | Water demin. | | Aqua dem. |
| 0.10 | D-Panthenol USP | (1) | Panthenol |
| 0.10 | Dow Corning 556 | (16) | Phenyl Trimethicone |
| 0.10 | Perfume oil | | |
| 40.00 | DME | — | Dimethylether |

Suppliers
(1) BASF Aktiengesellschaft
(16) Dow Corning Corporation

Aerosol spray without the addition of water containing polymer as in Ex. 1g) to 11) and Luviflex Silk

| % | Raw material | Supplier | INCI |
|---|---|---|---|
| 7.00 | Polymer 1g) to 11) | (1) | |
| 4.00 | Luviflex Silk. | (1) | PEG/PPG-25/25 Dimethi- |
| | | | cone/Acrylates Copolymer |
| 0.47 | AMP | (56) | Aminomethyl Propanol |
| 48.23 | Ethanol absolute | | Alcohol |
| 0.10 | Palatinol A | (1) | Diethyl Phthalate |
| 0.10 | D-Panthenol USP | (1) | Panthenol |
| 0.10 | Perfume oil | | |
| 10.00 | Propane/butane | — | Propane/Butane |
| 30.00 | DME | — | Dimethylether |

Suppliers
(1) BASF Aktiengesellschaft
(56) Angus Chemical Company

Aerosol spray without the addition of water containing polymer as in Ex. 1g) to 11) and Amphomer

| % | Raw material | Supplier | INCI |
|---|---|---|---|
| 10.00 | Polymer 1g) to 11) | (1) | |
| 1.00 | Amphomer 28-4910 | (72) | Acrylates Copolymer |
| 0.17 | AMP | (56) | Aminomethyl Propanol |
| 43.53 | Ethanol absolute | | Alcohol |
| 0.10 | Dow Corning 193 | (16) | PEG-12 Dimethicone |
| 0.10 | Dow Corning 556 | (16) | Phenyl Trimethicone |
| 0.10 | Perfume oil | | |
| 45.00 | DME | — | Dimethylether |

Suppliers
(1) BASF Aktiengesellschaft
(16) Dow Corning Corporation
(56) Angus Chemical Company
(72) National Starch & Chemical Limited

Mixing procedures

Pump Mousse Hair Setting Composition

| | | |
|---|---|---|
| 3.00 | Polymer 1g) | |
| 1.00 | Luviquat Mono CP | Hydroxyethyl Cetyl- |
| | | dimonium Phosphate |
| 0.20 | Cremophor A 25 | Ceteareth-25 |
| 0.40 | Perfume oil PC 910.781/Cremophor | |
| 95.40 | Water demin. | Aqua dem. |
| q.s. | Preservative | |

Preparation:

Prepare a uniform mixture from the components and bottle in a pump mousse bottle.

Pump Spray

| A | q.s. | Cremophor CO 40 | PEG-40 Hydrogenated |
|---|---|---|---|
| | | | Castor Oil |
| | q.s. | Perfume oil | |
| | 75.50 | Water demin. | Aqua dem. |
| | 7.30 | Polymer 1h) | |
| B | 1.00 | 1,2-Propylene glycol Care | Propylene Glycol |
| | 0.20 | Uvinul P 25 | PEG-25 PABA |
| | 1.00 | Luviquat HM 552 | Polyquaternium-16 |
| | 15.00 | Ethanol 96% | Alcohol |

Preparation:

Stir phase A. Add the components of phase B one after the other and distribute uniformly. Then bottle everything.

Styling Water

| | | | |
|---|---|---|---|
| A | 0.70 | Cremophor CO 40 | PEG-40 Hydrogenated |
| | | | Castor Oil |
| | 0.20 | Perfume oil | |
| | 75.10 | Water demin. | Aqua dem. |
| | 7.30 | Polymer 1i) | |
| B | 1.00 | 1,2-Propylene glycol Care | Propylene Glycol |
| | 0.50 | Luviquat Care | Polyquaternium-44 |
| | 0.20 | Uvinul P 25 | PEG-25 PABA |
| | 15.00 | Ethanol 96% | Alcohol |

Preparation:

Stir phase A. Add the components of phase B one after the other and distribute uniformly. Then bottle everything.

Hair Mousse

| | | | |
|---|---|---|---|
| A | 0.70 | Cremophor CO 40 | PEG-40 Hydrogenated |
| | | | Castor Oil |
| | 0.20 | Perfume oil | |
| | 78.50 | Water demin. | Aqua dem. |
| B | 0.50 | Luviquat Mono LS | Cocotrimonium Metho- |
| | | | sulfate |
| | 6.70 | Polymer 1g) | |
| | 2.50 | Luviquat Hold | Polyquaternium-46 |
| | 0.20 | Uvinul P 25 | PEG-25 PABA |
| | 0.50 | Pluracare E 400 | PEG-8 |
| | 0.20 | Cremophor A 25 | Ceteareth-25 |
| | q.s. | Preservative | |
| C | 10.00 | Propane/butane 3.5 bar (20° C.) | Propane/Butane |

Preparation:

Stir phase A. Add the components of phase B one after the other and distribute uniformly. Bottle with phase C.

Styling Mousse

| | | | |
|---|---|---|---|
| A | 2.00 | Luviquat Mono LS | Cocotrimonium Methosulfate |
| | q.s. | Perfume oil | |
| B | 62.85 | Water demin. | Aqua dem. |
| | 7.00 | Polymer 1h) | |
| | 2.00 | Luviquat PQ 11 | Polyquaternium-11 |
| | 0.20 | Cremophor A 25 | Ceteareth-25 |
| | 0.50 | D-Panthenol USP | Panthenol |
| | 0.05 | Uvinul MS 40 | Benzophenone-4 |
| | 0.20 | Dow Corning 949 Cationic | |
| | 15.00 | Ethanol 96% | Alcohol |
| C | 0.20 | Natrosol 250 HR | Hydroxyethylcellulose |
| D | 10.00 | Propane/butane | Propane/Butane |
| | | 3.5 bar (20° C.) | |

Preparation:

Mix phase A. Add the components of phase B one after the other and mix. Add phase C and stir until uniformlyd istributed . Adjust the pH to 6-7. Bottle with phase D.

Setting Foam

| | | | |
|---|---|---|---|
| A | 2.00 | Luviquat Mono LS | Cocotrimonium |
| | | | Methosulfate |
| | q.s. | Perfume oil | |
| B | 83.13 | Water demin. | Aqua dem. |
| | 0.47 | AMP | Aminomethyl Propanol |
| | 0.20 | Preservative | |
| | 0.20 | Abil B 8843 | Dimethicone Copolyol |
| C | 4.00 | Polymer 1l) | |
| D | 10.00 | Propane/butane 3.5 bar (20° C.) | Propane/Butane |

Preparation:

Mix phase A. Weigh in phase B and dissolve until clear. Stir phase B into phase A. Add phase C and stir. Bottle with phase D.

Wetlook Setting Foam

| | | | |
|---|---|---|---|
| A | 3.00 | Luviquat Mono LS | Cocotrimonium Methosulfate |
| | 0.20 | Perfume oil | |
| B | 78.80 | Water demin. | Aqua dem. |
| C | 5.00 | Glycerol 87% | Glycerin |
| | q.s. | Preservative | |
| | 3.00 | Polymer 1l) | |
| D | 10.00 | Propane/butane 3.5 bar | Propane/Butane |
| | | (20° C.) | |

Preparation:

Mix phase A. Stir phase B into phase A. Add phase C. Bottle with phase D.

Foam Conditioner

| | | |
|---|---|---|
| 5.00 | Luviquat PQ 11 | Polyquaternium-11 |
| 5.00 | Polymer 1f) | |
| 0.50 | Luviquat Mono CP | Hydroxyethyl Cetyl- |
| | | dimonium Phosphate |
| 10.00 | Ethanol abs. | Alcohol |
| 0.40 | Perfume oil “Carina”/Cremophor RH | |
| q.s. | Preservative | |
| 69.10 | Water demin. | Aqua dem. |
| 10.00 | Propane/butane | Propane/Butane |

Preparation:

Weigh everything together, stir until homogeneously distributed, bottle.

Sheen Hair Wax

| | | |
|---|---|---|
| 5.00 | Luvitol EHO | Cetearyl Octanoate |
| 5.00 | Castor oil | Castor (Ricinus |
| | | Communis) Oil |
| 17.00 | Vaseline | Petrolatum |
| 7.00 | TeCero wax 103 K | Microcrystalline Wax |
| 6.00 | Beeswax 3044 PH | Bees Wax |
| 5.00 | Polymer 1i) (anhydrous) | |
| 3.00 | Uvinul MBC 95 | 4-Methylbenzylidene |
| | | Camphor |
| 2.00 | Uvinul BMBM | Butyl Methoxydibenzoyl- |
| | | methane |
| 0.10 | Phytantriol | Phytantriol |
| 0.50 | Phenoxyethanol | Phenoxyethanol |
| 48.40 | Paraffin oil, high viscosity | Mineral Oil |
| .00 | Dow Corning 556 fluid | Phenyl Trimethicone |
| q.s. | Perfume oil | |

Preparation:

Weigh in the components of phase A and melt.

Color Balsam

| | | | |
|---|---|---|---|
| A | 1.50 | Cremophor A 6 | Ceteareth-6, Stearyl Alcohol |
| | 1.50 | Cremophor A 25 | Ceteareth-25 |
| | 3.00 | Cetylstearyl alcohol | Cetearyl Alcohol |
| | 6.00 | Luvitol EHO | Cetearyl Octanoate |
| | 0.30 | Phytantriol | Phytantriol |
| B | 7.70 | Luviquat Care | Polyquaternium-44 |
| | 6.00 | Polymer 1l) | |
| | 2.00 | 1,2-Propylene glycol Care | Propylene Glycol |
| | 1.00 | D-Panthenol USP | Panthenol |
| | q.s. | Preservative | |
| | 70.87 | Water demin. | Aqua dem. |
| C | 0.05 | Basic Violet 14 | C.I. 42510, Basic Violet 14 |
| | 0.08 | Basic Red 76 | C.I. 12245, Basic Red 76 |
| | q.s. | Perfume oil | |
| | q.s. | Citric acid | Citric Acid |

Preparation:

Heat phases A and B separately to about 80° C. Stir phase B into phase A with homogenization, briefly after-homogenize. Cool to about 40° C., add phase C and briefly homogenize again. Adjust the pH to 6 to 7.

Hair Repair Treatment

| | | | |
|---|---|---|---|
| A | 0.20 | Luvitol EHO | Cetearyl Octanoate |
| | 3.00 | Polymer 1f) | |
| | 0.10 | Phytantriol | Phytantriol |
| | 2.00 | Cremophor CO 40 | PEG-40 Hydrogenated |
| | | | Castor Oil |
| B | q.s. | Perfume oil | |
| | 2.00 | Luviquat Mono LS | Cocotrimonium Methosulfate |
| C | 79.70 | Water demin. | Aqua dem. |
| D | 2.00 | Luviquat FC 905 | Polyquaternium-16 |
| | 1.00 | Silicone oil SF 1288 | Dimethicone Copolyol |
| | q.s. | Preservative | |
| | 10.00 | Ethanol 96% | Alcohol |
| | q.s. | Citric acid | Citric Acid |

Preparation:

Separately mix the phases A and B. Stir phase C into phase B. Stir the solution of phases B and C into phase A. Add phase D and stir until thickened. Adjust the pH to 4 to 5.

Hair Gum

| | | | |
|---|---|---|---|
| A | 0.50 | Glucamate SSE-20 | PEG-20 Methyl Glucose |
| | | | Sesquistearate |
| | q.s. | Cremophor CO 40 | PEG-40 Hydrogenated |
| | | | Castor Oil |
| | q.s. | Perfume oil | |
| | 30.00 | Water demin. | Aqua dem. |
| B | 10.00 | Luviquat Hold | Polyquaternium-46 |
| | 2.00 | Luviskol K 90 | PVP |
| | 6.00 | Polymer 2a) | |
| | 0.30 | Germall 115 | Imidazolidinyl Urea |
| | 0.10 | Euxyl K 100 | Benzyl Alcohol, Methyl- |
| | | | chloroisothiazolinone, |
| | | | Methylisothiazone |
| | 0.50 | D-Panthenol USP | Panthenol |
| | 5.00 | Pluracare E 6000 | PEG 90 |
| | 3.00 | 1,2-Propylene glycol Care | Propylene Glycol |
| | 40.10 | Water demin. | Aqua dem. |
| C | 2.50 | Natrosol 250 HR | Hydroxyethylcellulose |

Preparation:

Solubilize phase A. Dissolve phase B and stir into phase A. Stir phase C into the solution of phases A and B.

Silky Hair Cocktail

| | | | |
|---|---|---|---|
| A | 3.00 | Luvigel EM | Caprylic/Capric Triglyceride, |
| | | | Acrylates Copolymer |
| | 3.00 | Polymer 2a) (anhydrous) | |
| | 0.50 | Wacker Belsil DMC 6031 | Dimethicone Copolyol |
| | 2.00 | Wacker Belsil DM 1000 | Dimethicone |
| | 3.00 | Wacker Belsil CM 1000 | Cyclomethicone, Dimethiconol |
| | 2.00 | Wacker Belsil ADM 6057E | Amodimethicone, Cetrimonium |
| | | | Chloride, Trideceth-10 |
| | 2.00 | Wacker Belsil PDM 200 | Phenyl Trimethicone |
| | 1.00 | Macadamia nut oil | Macadamia (Ternifolia) |
| | | | Nut Oil |
| | 0.50 | Vitamin E acetate | Tocopheryl Acetate |
| | 1.00 | Cremophor CO 40 | PEG-40 Hydrogenated |
| | | | Castor Oil |
| | q.s. | Perfume oil | |
| B | 77.54 | Water demin. | Aqua dem. |
| | 0.46 | AMP | Aminomethyl Propanol |
| | 4.00 | Luviflex Silk | PEG/PPG-25/25 Dimethicone/ |
| | | | Acrylates Copolymer |
| | q.s. | Preservative | |

Preparation:

Mix the components of phase A. Dissolve phase B. Stir phase B into phase A with homogenization.

Oil Sheen Moisturizer

| | | | |
|---|---|---|---|
| A | 2.00 | Cetyl alcohol | Cetyl Alcohol |
| | 1.00 | Solan ELD | PEG-75 Lanolin |
| | 4.00 | Glycerol monostearate | Glyceryl Stearate |
| | 1.00 | Cremophor A 25 | Ceteareth-25 |
| | 4.00 | Luvitol EHO | Cetearyl Octanoate |
| B | 10.00 | Glycerol 87% | Glycerin |
| | 5.00 | polymer 2b) | |
| | 2.00 | 1,2-Propylene glycol Care | Propylene Glycol |
| | 1.00 | Luviquat Mono LS | Cocotrimonium Methosulfate |
| | 1.50 | Silicone microemulsion | Trimethylsilylamodimethi- |
| | | | cone, SM 2115 Octoxynol-40, |
| | | | Isolaureth-6, Glycerin |
| | 1.00 | Cremophor PS 20 | Polysorbate 20 |
| | 67.00 | Water demin. | Aqua dem. |
| C | 0.50 | D-Panthenol USP | Panthenol |
| | q.s. | Preservative | |
| | q.s. | Perfume oil | |
| | q.s. | Citric acid | Citric Acid |

Preparation:

Heat phases A and B separately to about 80° C. Stir phase B into phase A and homogenize. Cool to about 40° C., add phase C and homogenize well again.

Setting Cream High Gloss

| | | | |
|---|---|---|---|
| A | 5.00 | Cetyl alcohol | Cetyl Alcohol |
| | 10.00 | Tegin | Glyceryl Stearate SE |
| | 5.00 | Isopropyl myristate | Isopropyl Myristate |
| | q.s. | Preservative | |
| | 1.00 | Dow Corning 200 fluid | Dimethicone |
| B | 5.00 | Glycerol 87% | Glycerin |
| | 5.00 | Polymer 2b) | |
| | 0.20 | Edeta BD | Disodium EDTA |
| | 2.00 | Luviskol K 30 | PVP |
| | 66.80 | Water demin. | Aqua dem. |
| C | q.s. | Perfume oil | |

Preparation:

Heat phases A and B separately to about 80° C. Stir phase B into phase A and homogenize. Cool to about 40° C., add phase C and homogenize briefly again.

Permanent Wave

| | | | |
|---|---|---|---|
| A | 70.95 | Water demin. | Aqua dem. |
| | 3.00 | Polymer 2c) | |
| | 0.20 | Tego Betain L 7 | Cocamidopropyl Betaine |
| | 0.20 | Cremophor PS 20 | Polysorbate 20 |
| | 1.25 | Luviquat FC 905 | Polyquaternium-16 |
| | 0.20 | Edeta BD | Disodium EDTA |
| | 0.20 | Natrosol 250 HR | Hydroxyethylcellulose |
| B | 8.00 | Thioglycolic acid 80% | Thioglycolic Acid |
| C | 11.00 | Ammonium solution 25% | Ammonium Hydroxide |
| D | 5.00 | Ammonium carbonate | Ammonium Carbonate |

Preparation:

Weigh in the components of phase A and mix. Stir phase B into phase A.

Neutralizer for Permanent Wave

| | | | |
|---|---|---|---|
| A | 1.00 | Cremophor CO 40 | PEG-40 Hydrogenated |
| | | | Castor Oil |
| | 0.20 | Perfume oil | |
| | 2.00 | Polymer 2c) | |
| | 91.60 | Water demin. | Aqua dem. |
| B | 0.20 | Tego Betain L 7 | Cocamidopropyl Betaine |
| | 0.20 | Cremophor A 25 | Ceteareth-25 |
| | 2.50 | Luviquat FC 905 | Polyquaternium-16 |
| | q.s. | Preservative | |
| C | 2.30 | Hydrogen peroxide 30% | Hydrogen Peroxid |
| D | q.s. | Phosphoric acid 85% | Phosphoric Acid |

Preparation:

Solubilize phase A. Add the components of phase B one after the other and mix. Add phase C and stir again. Adjust the pH to 3.0 to 3.5.

1-5. (canceled)

6. A cosmetic preparation which comprises polymer blend which comprises
1 to 98.9% by weight of vinylcaprolactam (monomer A),
1 to 98.9% by weight of vinylpyrrolidone (monomer B),
0.1 to 5% by weight of vinylimidazole (monomer C),
0 to 10% by weight of monomer D,
0 to 10% by weight (based on the total amount of monomer) of polymer E,
where the weight ratio of monomer C to monomer B is less than 1:12.

7. The composition according to claim 6, where the polymer comprises
30 to 59% by weight monomer A
40 to 69% by weight of monomer B
1 to 4.9% by weight of monomer C
0 to 10% by weight of monomer D
0 to 10% by weight (based on the total amount of monomer) of polymer E.

8. The composition according to claim 6, wherein said composition is used in hair cosemetics.

9. The composition according to claim 6, wherein said composition is used in hair-setting compositions or hair shampoo.

10. The composition according to claim 6, wherein said composition is used in skin cosmetic preparations.

11. A hair-setting preparation, in the form of a foam, mousse, spray or gel, where the active constituent used is a polymer according to claim 6.

12. The composition according to claim 6, where the polymer comprises
35 to 50% by weight monomer A
49 to 62% by weight monomer B
1 to 3% by weight monomer C
0 to 10% by weight of monomer D
0 to 10% by weight (based on the total amount of monomer) of polymer E,
where the weight ratio of monomer C to monomer B is less than or equal to 1:14.

13. The composition according to claim 12, wherein the weight ratio of monomer C to monomer B is less than or equal to 1:23.
